(19) United States
(12) Patent Application Publication
(10) Pub. No.: US 2026/0335335 A1
(43) Pub. Date: Oct. 29, 2026

(54) PHARMACEUTICAL FORMULATION COMPRISING A COATED TABLET

(71) Applicants: Genentech, Inc., South San Francisco, CA (US); Hoffmann-La Roche Inc., Little Falls, NJ (US)

(72) Inventors: Dawen Kou, Palo Alto, CA (US); Caroline Maierhofer, Muttenz (CH); Chen Mao, Foster City, CA (US); Avinash Murthy, Basel (CH); Karthik Nagapudi, San Ramon, CA (US)

(73) Assignees: Genentech, Inc., South San Francisco, CA (US); Hoffmann-La Roche Inc., Little Falls, NJ (US)

(21) Appl. No.: 19/535,600

(22) Filed: Feb. 10, 2026

Related U.S. Application Data
(63) Continuation of application No. PCT/EP2024/072652, filed on Aug. 9, 2024.
(60) Provisional application No. 63/532,164, filed on Aug. 11, 2023.

(51) Int. Cl. A61K 31/553; A61K 9/28; A61K 47/24; A61K 47/26; A61K 47/38

(52) U.S. Cl. CPC A61K 31/553; A61K 9/2813; A61K 9/284; A61K 9/2853; A61K 9/2893; A61K 47/24; A61K 47/26; A61K 47/38

(57) ABSTRACT

The invention relates to pharmaceutical formulation in form of a pharmaceutical formulation comprising GDC-0077 (INAVOLISIB) or a salt thereof, the formulation having a colored coating.

### CROSS-REFERENCE TO RELATED APPLICATIONS

This application is a continuation of International Application No. PCT/EP2024/072652, filed Aug. 9, 2024, which claims benefit of priority to provisional U.S. Application No. 63/532,164, filed on Aug. 11, 2023, each of which is incorporated herein by reference in its entirety.

### FIELD OF THE INVENTION

The invention relates to a pharmaceutical formulation and in particular to a pharmaceutical formulation comprising GDC-0077 (INAVOLISIB) or a pharmaceutically acceptable salt thereof.

### BACKGROUND OF THE INVENTION

GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide has previously been described in WO2017/001645A1 (PCT/EP2016/065455):

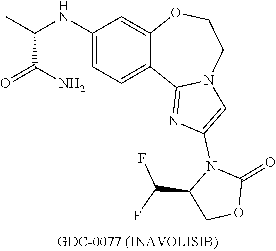 [SMILES: C[C@H](Nc1ccc2c(c1)OCCn1cc(N3C(=O)OC[C@H]3C(F)F)nc1-2)C(N)=O]

WO2018/109204A1 (PCT/EP2017/083143) relates to a method for preparing GDC-0077 (INAVOLISIB).

GDC-0077 (INAVOLISIB) is a benzoxazepin oxazolidinone compound with selective activity in modulating mutant forms of the PI3Kα (alpha) isoform.

Upregulation of the phosphoinositide-3 kinase (PI3K)/Akt signaling pathway is a common feature in most cancers (Yuan and Cantley (2008) Oncogene 27:5497-510). Genetic deviations in the pathway have been detected in many human cancers (Osaka et al (2004) Apoptosis 9:667-76) and act primarily to stimulate cell proliferation, migration, and survival. Activation of the pathway occurs following activating point mutations or amplifications of the PIK3CA gene encoding the p110α (alpha) PI3K isoforms (Hennessy et al (2005) Nat. Rev. Drug Discov. 4:988-1004). Genetic deletion or loss of function mutations within the tumor suppressor PTEN, a phosphatase with opposing function to PI3K, also increases PI3K pathway signaling (Zhang and Yu (2010) Clin. Cancer Res. 16:4325-30. These aberrations lead to increased downstream signaling through kinases such as Akt and mTOR and increased activity of the PI3K pathway has been proposed as a hallmark of resistance to cancer treatment (Opel et al (2007) Cancer Res. 67:735-45; Razis et al (2011) Breast Cancer Res. Treat. 128:447-56).

Phosphatidylinositol 3-Kinase (PI3K) is a major signaling node for key survival and growth signals for lymphomas and is opposed by the activity of the phosphatase PTEN. The phosphoinositide 3-dependent kinase (PI3K) signaling pathway is the most dysregulated pathway in hormone receptor positive breast cancer (HR+BC). The PI3K pathway is also dysregulated in aggressive forms of lymphoma (Abubaker (2007) Leukemia 21:2368-2370). Eight percent of DLBCL (diffuse large B-cell lymphoma) cancers have PI3CA (phosphatidylinositol-3 kinase catalytic subunit alpha) missense mutations and 37% are PTEN negative by immunohistochemistry test.

Phosphatidylinositol is one of a number of phospholipids found in cell membranes, and which participate in intracellular signal transduction. Cell signaling via 3′-phosphorylated phosphoinositides has been implicated in a variety of cellular processes, e.g., malignant transformation, growth factor signaling, inflammation, and immunity (Rameh et al (1999) J. Biol Chem. 274:8347-8350). The enzyme responsible for generating these phosphorylated signaling products, phosphatidylinositol 3-kinase (also referred to as PI 3-kinase or PI3K), was originally identified as an activity associated with viral oncoproteins and growth factor receptor tyrosine kinases that phosphorylate phosphatidylinositol (PI) and its phosphorylated derivatives at the 3′-hydroxyl of the inositol ring (Panayotou et al (1992) Trends Cell Biol 2:358-60).

Phosphoinositide 3-kinases (PI3K) are lipid kinases that phosphorylate lipids at the 3-hydroxyl residue of an inositol ring (Whitman et al (1988) Nature, 332:664). The 3-phosphorylated phospholipids (PIP3s) generated by PI3-kinases act as second messengers recruiting kinases with lipid binding domains (including plekstrin homology (PH) regions), such as Akt and PDK1, phosphoinositide-dependent kinase-1 (Vivanco et al (2002) Nature Rev. Cancer 2:489; Phillips et al (1998) Cancer 83:41).

The PI3 kinase family comprises at least 15 different enzymes sub-classified by structural homology and are divided into 3 classes based on sequence homology and the product formed by enzyme catalysis. The class I PI3 kinases are composed of 2 subunits: a 110 kd catalytic subunit and an 85 kd regulatory subunit. The regulatory subunits contain SH2 domains and bind to tyrosine residues phosphorylated by growth factor receptors with a tyrosine kinase activity or oncogene products, thereby inducing the PI3K activity of the p110 catalytic subunit which phosphorylates its lipid substrate. Class I PI3 kinases are involved in important signal transduction events downstream of cytokines, integrins, growth factors and immunoreceptors, which suggests that control of this pathway may lead to important therapeutic effects such as modulating cell proliferation and carcinogenesis. Class I PI3Ks can phosphorylate phosphatidylinositol (PI), phosphatidylinositol-4-phosphate, and phosphatidylinositol-4,5-biphosphate (PIP2) to produce phosphatidylinositol-3-phosphate (PIP), phosphatidylinositol-3,4-biphosphate, and phosphatidylinositol-3,4,5-triphosphate, respectively. Class II PI3Ks phosphorylate PI and phosphatidylinositol-4-phosphate. Class III PI3Ks can only phosphorylate PI. A key PI3-kinase isoform in cancer is the Class I PI3-kinase, p110α as indicated by recurrent oncogenic mutations in p110a (Samuels et al (2004) Science 304:554; U.S. Pat. Nos. 5,824,492; 5,846,824; 6,274,327). Other isoforms may be important in cancer and are also implicated in cardiovascular and immune-inflammatory disease (Workman P (2004) Biochem Soc Trans 32:393-396; Patel et al (2004) Proc. Am. Assoc. of Cancer Res. (Abstract LB-247) 95th Annual Meeting, March 27-31, Orlando, Florida, USA; Ahmadi K and Waterfield MD (2004) “Phosphoinositide 3-Kinase: Function and Mechanisms” Encyclopedia of Biological Chemistry (Lennarz W J, Lane M D eds) Elsevier/Academic Press), Oncogenic mutations of p110α (alpha) have been found at a significant frequency in colon, breast, brain, liver, ovarian, gastric, lung, and head and neck solid tumors. About 35-40% of hormone receptor positive (HR+) breast cancer tumors harbor a PIK3CA mutation. PTEN abnormalities are found in glioblastoma, melanoma, prostate, endometrial, ovarian, breast, lung, head and neck, hepatocellular, and thyroid cancers.

PI3 kinase (PI3K) is a heterodimer consisting of p85 and p110 subunits (Otsu et al (1991) Cell 65:91-104; Hiles et al (1992) Cell 70:419-29). Four distinct Class I PI3Ks have been identified, designated PI3K a (alpha), β (beta), 8 (delta), and y (gamma), each consisting of a distinct 110 kDha catalytic subunit and a regulatory subunit. Three of the catalytic subunits, i.e., p110 alpha, p110 beta and p110 delta, each interact with the same regulatory subunit, p85; whereas p110 gamma interacts with a distinct regulatory subunit, p101. The patterns of expression of each of these PI3Ks in human cells and tissues are distinct. In each of the PI3K alpha, beta, and delta subtypes, the p85 subunit acts to localize PI3 kinase to the plasma membrane by the interaction of its SH2 domain with phosphorylated tyrosine residues (present in an appropriate sequence context) in target proteins (Rameh et al (1995) Cell, 83:821-30; Volinia et al (1992) Oncogene, 7:789-93).

The PI3 kinase/Akt/PTEN pathway is an attractive target for cancer drug development since such agents would be expected to inhibit cellular proliferation, to repress signals from stromal cells that provide for survival and chemoresistance of cancer cells, to reverse the repression of apoptosis and surmount intrinsic resistance of cancer cells to cytotoxic agents. PI3K is activated through receptor tyrosine kinase signaling as well as activating mutations in the p110 catalytic subunit of PI3K, loss of the tumor suppressor PTEN, or through rare activating mutations in AKT.

GDC-0077 (INAVOLISIB) is a modulator of PI3Kα which is useful for treating cancers, particularly an inhibitor of PI3Kα that is selective for mutant PI3Kα expressing tumors relative to non-mutant PI3Kα expressing cells. GDC-0077 (INAVOLISIB) selectively inhibits the PI3Kα isoform relative to the PI3 KB, PI3K8, and PI3Ky isoforms, which may be expected to result in an enhanced therapeutic window and less side-effects. GDC-0077 (INAVOLISIB) is light sensitive.

### BRIEF SUMMARY OF THE INVENTION

An object of the present invention is to provide solid dosage forms in the form of stable tablet formulations of different strengths suitable for administration of GDC-0077 (INAVOLISIB) and methods to prepare the same. These formulations have a better manufacturability, stability and/or performance than previously described formulations.

One aspect of the invention provides a pharmaceutical formulation in a solid dosage form comprising a therapeutically effective amount of GDC-0077 (INAVOLISIB), or a pharmaceutically acceptable salt thereof, and a colored coating. In some embodiments, provided is a pharmaceutical formulation in a solid dosage form comprising a therapeutically effective amount of GDC-0077 (INAVOLISIB), or a pharmaceutically acceptable salt thereof, one or more intragranular excipients, one or more extragranular excipients, and a colored coating. In some embodiments, the solid dosage form is an immediate release tablet for oral administration.

Another aspect of the invention provides a process of making a pharmaceutical formulation described herein comprising the steps detailed herein.

Further provided is a method for treating cancer comprising administering to a patient in need thereof a pharmaceutical formulation comprising a therapeutically effective amount of GDC-0077 (INAVOLISIB) as described herein. In some embodiments, the patient has PIK3CA mutant, hormone receptor-positive, HER2-negative, locally advanced or metastatic breast cancer. In some embodiments, the patient has PIK3CA mutant, HER2-positive, locally advanced or metastatic breast cancer. In some embodiments, the method further comprises administering one or more additional anti-cancer agents.

Another aspect of the invention relates to a pharmaceutical formulation as described herein, for use in the treatment of cancer.

Another aspect of the invention relates to the use of a pharmaceutical formulation as described herein, in the manufacture of a medicament for the treatment of cancer.

In some of these embodiments, the cancer is a PIK3CA mutant, hormone receptor-positive, HER2-negative, locally advanced or metastatic breast cancer. In some of these embodiments, the cancer is a PIK3CA mutant, HER2-positive, locally advanced or metastatic breast cancer.

### BRIEF DESCRIPTION OF THE DRAWINGS

FIG. 1 is a flow chart depicting a manufacturing process of film-coated tablets according to Example 1.

FIG. 2 is a flow chart depicting a manufacturing process of film-coated tablets comprising a sandwiching step according to Example 2.

FIG. 3 is an image showing the visual appearance of pink tablets before and after 1×ICH light exposure.

FIG. 4 is an image showing the visual appearance of yellow tablets before and after 1×ICH light exposure.

FIG. 5 is an image showing the visual appearance of brown tablets before and after 1×ICH light exposure.

FIG. 6 is an image showing the visual appearance of brown tablets before and after 2× and 3×ICH light exposure.

FIG. 7 is an image showing the visual appearance after 3 and 7 days of direct sunlight exposure on a windowsill.

FIG. 8 is a graph showing the chromatograms of the tablets before and after 1×ICH light exposure.

### DETAILED DESCRIPTION OF THE INVENTION

The present invention provides stable tablet formulations of different strengths suitable for oral administration of GDC-0077 (INAVOLISIB).

In some embodiments, GDC-0077 (INAVOLISIB) has been formulated as 3 mg and 9 mg (amount of GDC-0077 (INAVOLISIB)) film-coated, immediate-release tablets for oral administration.

GDC-0077 (INAVOLISIB) (CAS RN 2060571-02-8) has the following structure

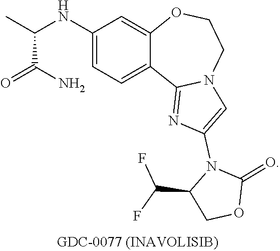 [SMILES: C[C@H](Nc1ccc2c(c1)OCCn1cc(N3C(=O)OC[C@H]3C(F)F)nc1-2)C(N)=O]

GDC-0077 (INAVOLISIB) degrades under light stress conditions. Accordingly, the present invention is directed to a solid dosage pharmaceutical formulation that protects the active pharmaceutical ingredient from light degradation. The formulation of this invention is a common blend, dose proportional film-coated tablet. In some embodiments, the solid dosage pharmaceutical formulation comprises a therapeutically effective amount of GDC-0077 (INAVOLISIB) that is between about 1 mg and about 20 mg, such as between about 3 mg and about 10 mg, such as about 3 mg, 4, mg, 5 mg, 6 mg, 7 mg, 8 mg, 9, mg, or 10 mg. In some embodiments, the solid dosage pharmaceutical formulation comprises a core comprising the therapeutically effective amount of GDC-0077 (INAVOLISIB) and further comprises a coating, preferably a color coating. The core comprising the therapeutically effective amount of GDC-0077 (INAVOLISIB) comprises between about 1% by wt. and about 20% by weight GDC-0077 (INAVOLISIB), such as between about 3% by wt. and about 10% by wt. In some embodiments of the present invention GDC-0077 (INAVOLISIB) is used as a pharmaceutically acceptable salt. In some embodiments of the present invention GDC-0077 (INAVOLISIB) is used as the free base compound.

### DEFINITIONS

The following definitions of the general terms used in the present description apply irrespectively of whether the terms in question appear alone or in combination with other groups.

The term “about” in reference to defined parameters, e.g. mg, mm, or wt. %, means either within plus or minus 10% of the provided value, or rounded to the nearest significant figure, in all cases inclusive of the provided value.

The expression “wt. %” refers to percent weight/weight percent/% w/w with x/100, e.g. mg/100 mg or g/100 g.

In this invention the term “one or more”, when used in relation to number of excipients, means one or any reasonable number of excipients. Unless otherwise stated, the upper limit is not particularly defined. The term is further discussed in this application in relation to specific components of the formulation.

The term “disintegrant” refers to excipients that enhance disintegration of a solid dosage form, such as by expansion when wet causing the tablet to break apart in the body and release the active ingredient for absorption. Non-limiting examples include cross-linked polymers like crospovidone, croscarmellose sodium, etc. and modified starches like sodium starch glycolate. A specific, non-limiting, example is Primojel™.

The term “filler” refers to excipients that fill out the size of a tablet by increasing the bulk volume. Fillers make it possible for the final product to have the proper volume for patient handling. Non-limiting examples of fillers are plant cellulose, lactose, starch, mannitol, etc. Specific, non-limiting, examples are lactose monohydrate like Pharmatose 200M, microcrystalline cellulose (MCC) like Avicel PH101, or Avicel PH102 and spray dried lactose like Fast Flo 316™. Fillers are typically not pharmaceutically active.

The term “binder” refers to excipients that hold the ingredients in a tablet together. Binders ensure that tablets and granules can be formed with required mechanical strength. Non-limiting examples of binders are polyvinlypyrrolidon (PV), hydroxypropyl methylcellulose (HPMC), hydroxypropylcellulose (HPC), cellulose, sugar alcohols like sorbitol, proteins like gelatin and polymers like PVP, e.g. copovidone (PVP/VA 64), PEG, etc. A specific, non-limiting, example is Povidone K30™.

The term “lubricant” refers to excipients that prevent ingredients from clumping together and from sticking to the tablet punches or capsule filling machine. Lubricants also ensure that tablet formation and ejection can occur with low friction between active ingredient and wall. Non-limiting examples of lubricants are minerals like talc or silica and fats like stearin, magnesium stearate, etc. A specific, non-limiting, example is magnesium stearate such as for example MF-2-V™.

The term “coating agent” refers to functional coating agents that is a group of substances that provides a coating to provide a barrier to protect components of the solid dosage form from the external environment prior to administration, e.g. providing protection from ambient moisture, light etc. Coating agents may comprise water soluble, pH independent complete dry powder film coating system containing polymer, plasticizer and pigment which allows for immediate disintegration when administered for fast, active release. A specific, non-limiting, example is Opadry II™.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like.

This invention relates to a pharmaceutical formulation in the form of solid dosage form comprising GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the structural formula:

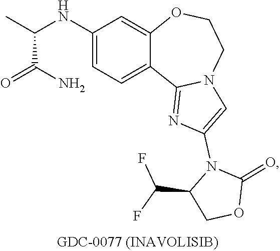 [SMILES: C[C@H](Nc1ccc2c(c1)OCCn1cc(N3C(=O)OC[C@H]3C(F)F)nc1-2)C(N)=O]

or a stereoisomer, geometric isomer, tautomer, or pharmaceutically acceptable salt thereof, wherein the pharmaceutical formulation comprises a colored coating.

This invention relates to a pharmaceutical formulation in the form of solid dosage form comprising GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the structural formula:

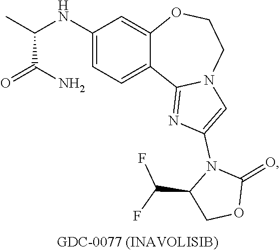 [SMILES: C[C@H](Nc1ccc2c(c1)OCCn1cc(N3C(=O)OC[C@H]3C(F)F)nc1-2)C(N)=O]

or a pharmaceutically acceptable salt thereof, wherein the pharmaceutical formulation comprises a colored coating.

This invention relates to a pharmaceutical formulation in the form of solid dosage form comprising free base GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the structural formula:

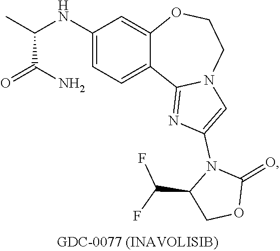 [SMILES: C[C@H](Nc1ccc2c(c1)OCCn1cc(N3C(=O)OC[C@H]3C(F)F)nc1-2)C(N)=O]

wherein the pharmaceutical formulation comprises a colored coating.

The pharmaceutical formulation in the form of a solid dosage form has some further advantages:
a) it provides a pharmaceutical formulation with improved light stability (physical and chemical stability);
b) the process of making such pharmaceutical formulation minimizes the loss of API during formulation and therefore reduces costs, resources and environmental impacts; and
c) the process of making such pharmaceutical formulation is robust and easily controlled and to be scaled up for clinical and commercial production.

A specific embodiment of present invention relates to a pharmaceutical formulation as described herein, wherein the pharmaceutical formulation is a tablet.

In a specific embodiment, the tablets are manufactured by blending (including pre-blending/sandwiching), sieving, compaction, tableting, and coating.

A specific embodiment of present invention relates to a pharmaceutical formulation as described herein, wherein the tablets have different forms, sizes and thicknesses. This makes use of the tablets by the patients easier and improves safety.

A specific embodiment of present invention relates to a pharmaceutical formulation as described herein, wherein the tablets have different forms, sizes and thicknesses depending on the amount of GDC-0077 (INAVOLISIB) comprised by the tablet.

A specific embodiment of present invention relates to a pharmaceutical formulation as described herein, wherein the tablets are round, oblong, concave, round convex, round bi-convex, or oval.

A specific embodiment of present invention relates to a pharmaceutical formulation as described herein, wherein the tablets are round bi-convex or oval.

A specific embodiment of present invention relates to a pharmaceutical formulation as described herein, wherein the tablet is round bi-convex and comprises about 3 mg GDC-0077 (INAVOLISIB).

A specific embodiment of present invention relates to a pharmaceutical formulation as described herein, wherein the tablet is oval and comprises about 9 mg GDC-0077 (INAVOLISIB).

A specific embodiment of the present invention relates to a pharmaceutical formulation as described herein, which comprises an engraving INA 3.

A specific embodiment of the present invention relates to a pharmaceutical formulation as described herein, which comprises an engraving INA 9.

A specific embodiment of the present invention relates to a pharmaceutical formulation as described herein, which comprises a debossing INA 3.

A specific embodiment of the present invention relates to a pharmaceutical formulation as described herein, which comprises a debossing INA 9.

A specific embodiment of present invention relates to a pharmaceutical formulation as described herein, wherein the tablet is for oral, buccal, sublingual, or vaginal administration.

A specific embodiment of present invention relates to a pharmaceutical formulation as described herein, wherein the tablet is for oral administration.

A specific embodiment of present invention relates to a pharmaceutical formulation as described herein, wherein the tablet is an immediate release or sustained release tablet.

A specific embodiment of present invention relates to a pharmaceutical formulation as described herein, wherein the tablet is an immediate release tablet.

A specific embodiment of present invention relates to a pharmaceutical formulation as described herein, wherein the tablet is an immediate release tablet for oral administration.

A specific embodiment of the present invention relates to a pharmaceutical formulation as described above, wherein the pharmaceutical formulation further comprises one or more excipients selected from the group consisting of binders, disintegrants, glidants, fillers and lubricants.

A specific embodiment of the present invention relates to a pharmaceutical formulation as described above, wherein the pharmaceutical formulation further comprises one or more excipients selected from the group consisting of fillers, disintegrants and lubricants.

A specific embodiment of the present invention relates to a pharmaceutical formulation as described above, wherein the pharmaceutical formulation comprises two fillers which can be the same (but may differ in physical nature, e.g. particle size) or different.

A specific embodiment of the present invention relates to a pharmaceutical formulation as described above, wherein the pharmaceutical formulation comprises two fillers which are different.

A specific embodiment of the present invention relates to a pharmaceutical formulation as described above, wherein the two different fillers are microcrystalline cellulose and lactose.

A specific embodiment of the present invention relates to a pharmaceutical formulation as described above, wherein the two different fillers are microcrystalline cellulose and spray dried lactose.

A specific embodiment of the present invention relates to a pharmaceutical formulation as described above, wherein the pharmaceutical formulation comprises one, preferably only one, disintegrant.

A specific embodiment of the present invention relates to a pharmaceutical formulation as described above, wherein the disintegrant (e.g. the only disintegrant) is sodium starch glycolate.

A specific embodiment of the present invention relates to a pharmaceutical formulation as described above, wherein the pharmaceutical formulation comprises one, preferably only one, lubricant.

A specific embodiment of the present invention relates to a pharmaceutical formulation as described above, wherein the lubricant (e.g. the only lubricant) is magnesium stearate.

A specific embodiment of this invention relates to a pharmaceutical formulation comprising intragranular and extragranular excipients.

A specific embodiment of this invention relates to a pharmaceutical formulation in form of solid dosage form comprising GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the formula:

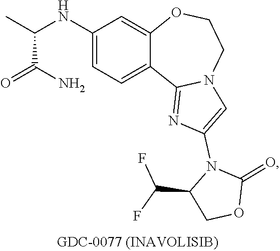 [SMILES: C[C@H](Nc1ccc2c(c1)OCCn1cc(N3C(=O)OC[C@H]3C(F)F)nc1-2)C(N)=O]

or a stereoisomer, geometric isomer, tautomer, or pharmaceutically acceptable salt thereof, wherein the pharmaceutical formulation comprises:
a therapeutically effective amount of GDC-0077 (INAVOLISIB),
one or more intragranular excipients,
one or more extragranular excipients, and
a colored coating.

A specific embodiment of this invention relates to a pharmaceutical formulation in form of solid dosage form comprising GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the formula:

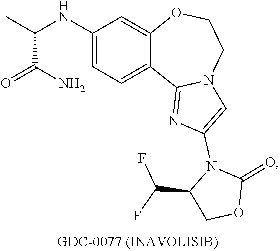 [SMILES: C[C@H](Nc1ccc2c(c1)OCCn1cc(N3C(=O)OC[C@H]3C(F)F)nc1-2)C(N)=O]

or a pharmaceutically acceptable salt thereof, wherein the pharmaceutical formulation comprises:
a therapeutically effective amount of GDC-0077 (INAVOLISIB),
one or more intragranular excipients,
one or more extragranular excipients, and
a colored coating.

A specific embodiment of this invention relates to a pharmaceutical formulation in form of solid dosage form comprising free base GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the formula:

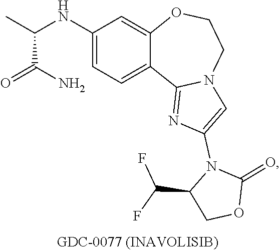 [SMILES: C[C@H](Nc1ccc2c(c1)OCCn1cc(N3C(=O)OC[C@H]3C(F)F)nc1-2)C(N)=O]

wherein the pharmaceutical formulation comprises:
a therapeutically effective amount of free base GDC-0077 (INAVOLISIB),
one or more intragranular excipients,
one or more extragranular excipients, and
a colored coating.

A specific embodiment of present invention relates to a pharmaceutical formulation as described above, wherein the pharmaceutical formulation is a tablet.

A specific embodiment of present invention relates to a pharmaceutical formulation as described above, wherein the tablet is for oral, buccal, sublingual, or vaginal administration.

A specific embodiment of present invention relates to a pharmaceutical formulation as described above, wherein the tablet is for oral administration.

A specific embodiment of present invention relates to a pharmaceutical formulation as described above, wherein the tablet is an immediate release or sustained release tablet.

A specific embodiment of present invention relates to a pharmaceutical formulation as described above, wherein the tablet is an immediate release tablet.

A specific embodiment of present invention relates to a pharmaceutical formulation as described above, wherein the tablet is an immediate release tablet for oral administration.

A specific embodiment of the present invention relates to a pharmaceutical formulation as described above, wherein the intragranular excipients comprise one or more (for example a total of one, two, three, four, or five) binders, disintegrants, glidants, fillers and lubricants.

A specific embodiment of the present invention relates to a pharmaceutical formulation as described above, wherein the intragranular excipients comprise one or more fillers, disintegrants and lubricants.

A specific embodiment of the present invention relates to a pharmaceutical formulation as described above, wherein the intragranular excipients comprise one or more fillers, disintegrants and lubricants, where one or more may be a total number of one, two, three, four, or five. There may be one or more of each excipient, e.g. two fillers.

A specific embodiment of the present invention relates to a pharmaceutical formulation as described above, wherein the pharmaceutical formulation comprises two, preferably only two, intragranular fillers.

A specific embodiment of the present invention relates to a pharmaceutical formulation as described above, wherein the two intragranular fillers are microcrystalline cellulose and lactose.

A specific embodiment of the present invention relates to a pharmaceutical formulation as described above, wherein the two intragranular fillers are microcrystalline cellulose and spray dried lactose.

A specific embodiment of the present invention relates to a pharmaceutical formulation as described above, wherein the pharmaceutical formulation comprises one, preferably only one, intragranular disintegrant.

A specific embodiment of the present invention relates to a pharmaceutical formulation as described above, wherein the intragranular disintegrant (e.g. the only intragranular disintegrant) is sodium starch glycolate.

A specific embodiment of the present invention relates to a pharmaceutical formulation as described above, wherein the pharmaceutical formulation comprises one, preferably only one, intragranular lubricant.

A specific embodiment of the present invention relates to a pharmaceutical formulation as described above, wherein the intragranular lubricant (e.g. the only intragranular lubricant) is magnesium stearate.

A specific embodiment of the present invention relates to a pharmaceutical formulation as described above, wherein the extragranular excipients comprise one or more (for example a total of one, two, three, four, or five) binders, disintegrants, glidants, fillers and lubricants.

A specific embodiment of the present invention relates to a pharmaceutical formulation as described above, wherein the extragranular excipients comprise one or more disintegrants and lubricants.

A specific embodiment of the present invention relates to a pharmaceutical formulation as described above, wherein the extragranular excipients comprise one or more (e.g. one two or three) disintegrants and lubricants. There may be one or more of each excipient, e.g. one disintegrant and one lubricant.

A specific embodiment of present invention relates to a pharmaceutical formulation as described herein, wherein the pharmaceutical formulation comprises one (preferably only one) extragranular disintegrant.

A specific embodiment of present invention relates to a pharmaceutical formulation as described herein, wherein the extragranular disintegrant (e.g. the only extragranular disintegrant) is sodium starch glycolate.

A specific embodiment of present invention relates to a pharmaceutical formulation as described herein, wherein the pharmaceutical formulation comprises one, preferably only one, extragranular lubricant.

A specific embodiment of the present invention relates to a pharmaceutical formulation as described above, wherein the extragranular lubricant (e.g. the only extragranular lubricant) is magnesium stearate.

A specific embodiment of present invention relates to a pharmaceutical formulation as described herein, wherein the colored coating is a water-soluble film coating. These coatings are very helpful film coating systems delivering high adhesion, quality finish and manufacturing efficiency.

A specific embodiment of present invention relates to a pharmaceutical formulation as described herein, wherein the colored coating is a water soluble and/or pH independent film coating.

A specific embodiment of present invention relates to a pharmaceutical formulation as described herein, wherein the colored coating is a water soluble and pH independent film coating.

A specific embodiment of present invention relates to a pharmaceutical formulation as described herein, where the colored coating comprises one or more components selected from polymers, plasticizers, metal oxides, colored pigments, and other additives as appropriate.

A specific embodiment of present invention relates to a pharmaceutical formulation as described herein, wherein the colored coating comprises polyvinylalcohol (which can be partially hydrolyzed).

A specific embodiment of present invention relates to a pharmaceutical formulation as described herein, wherein the colored coating comprises one or more components selected from polyvinylalcohol (which can be partially hydrolyzed), titanium oxides (e.g. titanium dioxide), colored pigments, polyethylene glycol and talc.

The colored coating can be applied by any appropriate technical method. In a specific embodiment of the present invention, the colored coating is applied using a perforated pan coater. In a specific embodiment of the present invention, the colored coating is applied using purified water as vehicle which is removed during coating.

A specific embodiment of present invention relates to a pharmaceutical formulation as described herein, wherein the colored coating comprises one or more titanium oxide(s).

A specific embodiment of present invention relates to a pharmaceutical formulation as described herein, wherein the colored coating comprises only one titanium oxide.

A specific embodiment of present invention relates to a pharmaceutical formulation as described herein, wherein the colored coating comprises one or more titanium oxide(s) and/or iron oxide(s).

A specific embodiment of present invention relates to a pharmaceutical formulation as described herein, wherein the colored coating comprises one or more titanium oxide(s) and no iron oxide(s).

A specific embodiment of present invention relates to a pharmaceutical formulation as described herein, wherein the colored coating comprises one or more titanium oxide(s) and/or iron oxide(s)

The combination of titanium oxide(s) and iron oxides may have a synergistic effect, for example providing a higher than additive level of protection from light for a formulation coated with a coating containing these components.

A specific embodiment of present invention relates to a pharmaceutical formulation as described herein, wherein, wherein the term “one or more” in relation to the iron oxides means one, two, or three.

A specific embodiment of present invention relates to a pharmaceutical formulation as described herein, wherein the colored coating comprises only one titanium oxide and one or more iron oxide(s).

A specific embodiment of present invention relates to a pharmaceutical formulation as described herein, wherein the titanium oxide is titanium dioxide.

A specific embodiment of present invention relates to a pharmaceutical formulation as described herein, wherein the colored pigments comprise one or more different iron oxide(s).

A specific embodiment of present invention relates to a pharmaceutical formulation as described herein, wherein the colored coating comprises titanium dioxide and one or more different iron oxide(s).

A specific embodiment of present invention relates to a pharmaceutical formulation as described herein, wherein the colored coating comprises titanium dioxide and one or more iron oxide(s) selected from iron oxide (black), iron oxide (red), and iron oxide (yellow).

A specific embodiment of present invention relates to a pharmaceutical formulation as described herein, wherein the colored coating comprises titanium dioxide and one or more iron oxide(s) selected from iron oxide (black) Fe₃O₄, iron oxide (red) Fe₂O₃, and iron oxide (yellow) FeH₃O₃.

A specific embodiment of present invention relates to a pharmaceutical formulation as described herein, wherein the colored pigments comprise one, two or three different iron oxides.

A specific preferred embodiment of present invention relates to a pharmaceutical formulation as described herein, wherein the colored pigments comprise only one iron oxide.

A specific preferred embodiment of present invention relates to a pharmaceutical formulation as described herein, wherein the colored pigments comprise two different iron oxides.

A specific embodiment of present invention relates to a pharmaceutical formulation as described herein, wherein the colored pigments comprise three different iron oxides.

A specific embodiment of present invention relates to a pharmaceutical formulation as described herein, where the iron oxide is one, two or three iron oxides selected from iron oxide (red), iron oxide (yellow) and iron oxide (black).

A specific embodiment of present invention relates to a pharmaceutical formulation as described herein, wherein the colored coating comprises the following (two separate embodiments, “Red” and “Pink” are defined):

### Red:

| | |
|---|---|
| Polyvinyl alcohol | Filming agent |
| Titanium dioxide | Opacifier |
| Macrogol/PEG | Plasticizer |
| Talc | Lubricant |
| Iron oxide red | Pigment |

### Pink:

| | |
|---|---|
| Polyvinyl alcohol | Filming agent |
| Titanium dioxide | Opacifier |
| Macrogol/PEG | Plasticizer |
| Talc | Lubricant |
| Iron oxide red | Pigment |
| Iron oxide yellow | Pigment |

A specific embodiment of present invention relates to a pharmaceutical formulation as described herein, wherein the colored coating consists of (wt. %):

| | |
|---|---|
| Polyvinylalcohol (partially hydrolyzed) | about 40.0% |
| Titanium dioxide | about 20.8% |
| Macrogol/Polyethylene glycol | about 20.2% |
| Talc | about 14.8% |
| Iron oxide (yellow) | about 3.3% |
| Iron oxide (red) | about 0.8% |
| Iron oxide (black) | about 0.1% |

A specific embodiment of present invention relates to a pharmaceutical formulation as described herein, wherein the colored coating consists of (wt. %):

| | |
|---|---|
| Polyvinylalcohol (partially hydrolyzed) | about 40.0% |
| Titanium dioxide | about 25.0% |
| Macrogol/Polyethylene glycol | about 20.2% |
| Talc | about 14.8% |

A specific embodiment of present invention relates to a pharmaceutical formulation as described herein, wherein the colored coating consists of (wt. %):

| | |
|---|---|
| Polyvinylalcohol (partially hydrolyzed) | about 40.0% |
| Titanium dioxide | about 24.7% |
| Macrogol/Polyethylene glycol | about 20.2% |
| Talc | about 14.8% |
| Iron oxide (yellow) | about 0.7% |

A specific preferred embodiment of present invention relates to a pharmaceutical formulation as described herein, wherein the colored coating consists of (wt. %):

| | |
|---|---|
| Polyvinylalcohol (partially hydrolyzed) | about 40.0% |
| Titanium dioxide | about 10.0% |
| Macrogol/Polyethylene glycol | about 20.2% |
| Talc | about 14.8% |
| Iron oxide (red) | about 15.0% |

A specific preferred embodiment of present invention relates to a pharmaceutical formulation as described herein, wherein the colored coating consists of (wt. %):

| | |
|---|---|
| Polyvinylalcohol (partially hydrolyzed) | about 40.0% |
| Titanium dioxide | about 22.1% |
| Macrogol/Polyethylene glycol | about 20.2% |
| Talc | about 14.8% |
| Iron oxide (red) | about 1.5% |
| Iron oxide (yellow) | about 1.4% |

A specific embodiment of present invention relates to a pharmaceutical formulation as described herein, wherein the colored coating is Opadry II™.

A specific embodiment of present invention relates to a pharmaceutical formulation as described herein, wherein the Opadry II™ coating is selected from Opadry II™ (white), Opadry II™ (yellow), Opadry II™ (pink), Opadry II™ (red), and Opadry II™ (brown).

A specific embodiment of present invention relates to a pharmaceutical formulation as described herein, wherein the colored coating is Opadry II™ (white).

A specific embodiment of present invention relates to a pharmaceutical formulation as described herein, wherein the colored coating is Opadry II™ (yellow).

A specific preferred embodiment of present invention relates to a pharmaceutical formulation as described herein, wherein the colored coating is Opadry II™ (pink).

A specific preferred embodiment of present invention relates to a pharmaceutical formulation as described herein, wherein the colored coating is Opadry II™ (red).

A specific embodiment of present invention relates to a pharmaceutical formulation as described herein, wherein the colored coating is Opadry II™ (brown).

A coloured coating prevents transmission of certain wavelengths of light, the wavelengths depending on the colour of the coating, and therefore protects any underlying formulation from such light. This may provide an advantage in that the components of the formulation, particularly GDC-0077 (INAVOLISIB), are protected from such wavelengths of light and any associated degradation that such exposure may cause.

In preferred embodiments the colored coating on the present pharmaceutical formulations provides a coating that has low light transmissibility. In particular, low transmissibility to one or more of visible or ultraviolet light. For example, the colored coating has a light transmissibility of lower than about 25%, preferably lower than about 20%, preferably lower than about 15%, preferably lower than about 10%, preferably lower than about 5%, preferably lower than about 2%, preferably lower than about 1%. In some cases, the colored coating provides a complete barrier to light transmission, i.e. 0% light transmissibility. This low light transmissibility protects the active GDC-0077 (INAVOLISIB) from light exposure and any associated degradation.

An additional advantage of oxide colored pigments of the present invention are good chemical and light stability, small particle size, reduced coating time, little batch-to-batch colour variation (stable and consistent product performance), low cost and excellent opacity. Further additional advantages may include higher film adhesion, lower level of water permeation, superior oxygen barrier protection and/or greater aqueous coating uniformity.

Any colored coating similar, equivalent or the same as the afore-mentioned coatings can be used if they have similar, equivalent, or the same light protective properties.

A specific embodiment of the present invention relates to a pharmaceutical formulation as described herein, wherein the pharmaceutical composition comprises 3 mg GDC-0077 (INAVOLISIB) and the colored coating is red.

A specific embodiment of the present invention relates to a pharmaceutical formulation as described herein, wherein the pharmaceutical composition comprises 9 mg GDC-0077 (INAVOLISIB) and the colored coating is pink.

A specific embodiment of present invention relates to a pharmaceutical formulation as described herein, wherein the film-coated tablet comprises about 2-6 wt. % colored coating.

A specific embodiment of present invention relates to a pharmaceutical formulation as described herein, wherein the film-coated tablet comprises about 4 wt. % colored coating.

A specific embodiment of present invention relates to a pharmaceutical formulation as described herein, wherein the weight of the film-coating per tablet is in the range of about 3 mg to about 20 mg

A specific embodiment of present invention relates to a pharmaceutical formulation as described herein, wherein the weight of the film-coating per tablet is in the range of about 3 mg to about 15 mg.

A specific embodiment of present invention relates to a pharmaceutical formulation as described herein, wherein the weight of the film-coating per tablet is about 4 mg or about 12 mg.

A specific embodiment of present invention relates to a pharmaceutical formulation as described herein, wherein the weight of the film-coating per tablet is about 4 mg and the tablet comprises about 3 mg GDC-0077 (INAVOLISIB).

A specific embodiment of present invention relates to a pharmaceutical formulation as described herein, wherein the weight of the film-coating per tablet is about 12 mg and the tablet comprises about 9 mg GDC-0077 (INAVOLISIB).

A specific embodiment of present invention relates to a pharmaceutical formulation as described herein, wherein the weight ratio of the film-coating per tablet and the tablet is about 4 wt. %.

A specific embodiment of present invention relates to a pharmaceutical formulation as described herein, wherein the thickness of the colored coating is in the range of about 0.04 mm to about 0.12 mm.

A specific embodiment of present invention relates to a pharmaceutical formulation as described herein, wherein the thickness of the colored coating is in the range of about 0.05 mm to about 0.1 mm.

A specific embodiment of present invention relates to a pharmaceutical formulation as described herein, wherein the thickness of the colored coating is in the range of about 0.06 mm to about 0.09 mm.

A specific embodiment of present invention relates to a pharmaceutical formulation as described herein, wherein the pharmaceutical formulation is a round bi-convex or oval tablet.

A specific embodiment of present invention relates to a pharmaceutical formulation as described herein, wherein the pharmaceutical formulation is round bi-convex tablet.

A specific embodiment of present invention relates to a pharmaceutical formulation as described herein, wherein the diameter of the round bi-convex tablet is in the range of about 3 mm to about 15 mm.

A specific embodiment of present invention relates to a pharmaceutical formulation as described herein, wherein the diameter of the round bi-convex tablet is in the range of about 3 mm to about 8 mm.

A specific embodiment of present invention relates to a pharmaceutical formulation as described herein, wherein the diameter of the round bi-convex tablet is about 6 mm.

A specific embodiment of present invention relates to a pharmaceutical formulation as described herein, wherein the maximum thickness of the core of the round bi-convex tablet is in the range of about 3 mm to about 5 mm.

A specific embodiment of present invention relates to a pharmaceutical formulation as described herein, wherein the maximum thickness of the core of the round bi-convex tablet is in the range of about 3 mm to about 4 mm.

A specific embodiment of present invention relates to a pharmaceutical formulation as described herein, wherein the maximum thickness of the core of the round bi-convex tablet is in the range of about 3 mm to about 3.8 mm

A specific embodiment of present invention relates to a pharmaceutical formulation as described herein, wherein the maximum thickness of the core of the round bi-convex tablet is about 3.4 mm.

A specific embodiment of present invention relates to a pharmaceutical formulation as described herein, wherein the core of the round bi-convex tablet has a diameter of about 6 mm and a maximum thickness of about 3.4 mm.

A specific embodiment of present invention relates to a pharmaceutical formulation as described herein, wherein the core of the round bi-convex tablet has a diameter of about 6 mm, a maximum thickness of about 3.4 mm and comprises about 3 mg GDC-0077 (INAVOLISIB).

A specific embodiment of present invention relates to a pharmaceutical formulation as described herein, wherein the pharmaceutical formulation is an oval tablet.

A specific embodiment of present invention relates to a pharmaceutical formulation as described herein, wherein the core dimensions of the oval tablet are in the range of about 10 mm to about 15 mm by about 5 mm to about 8 mm.

A specific embodiment of present invention relates to a pharmaceutical formulation as described herein, wherein the core dimensions of the oval tablet are in the range of about 12 to about 14 mm by about 5 to about 7 mm.

A specific embodiment of present invention relates to a pharmaceutical formulation as described herein, wherein the core dimensions of the oval tablet are about 13 mm by about 6 mm.

A specific embodiment of present invention relates to a pharmaceutical formulation as described herein, wherein thickness of the core of the oval tablet is in the range of about 3 mm to about 6 mm.

A specific embodiment of present invention relates to a pharmaceutical formulation as described herein, wherein thickness of the core of the oval tablet is in the range of about 3 mm to about 5 mm.

A specific embodiment of present invention relates to a pharmaceutical formulation as described herein, wherein thickness of the core of the oval tablet is in the range of about 4 mm to about 5 mm.

A specific embodiment of present invention relates to a pharmaceutical formulation as described herein, wherein thickness of the core of the oval tablet is about 4.5 mm.

A specific embodiment of present invention relates to a pharmaceutical formulation as described herein, wherein the core dimensions of the oval tablet are about 13 mm by about 6 mm, the thickness is about 4.4 mm and the tablet comprises about 9 mg GDC-0077 (INAVOLISIB).

A specific embodiment of present invention relates to a pharmaceutical formulation as described herein, wherein the pharmaceutical formulation comprises about 2 to about 12 mg GDC-0077 (INAVOLISIB).

A specific embodiment of present invention relates to a pharmaceutical formulation as described herein, wherein the pharmaceutical formulation comprises about 2 to about 10 mg GDC-0077 (INAVOLISIB).

A specific embodiment of present invention relates to a pharmaceutical formulation as described herein, wherein the pharmaceutical formulation comprises about 3 or about 9 mg GDC-0077 (INAVOLISIB).

A specific embodiment of present invention relates to a pharmaceutical formulation as described herein, wherein the pharmaceutical formulation comprises about 3 mg GDC-0077 (INAVOLISIB).

A specific embodiment of present invention relates to a pharmaceutical formulation as described herein, wherein the pharmaceutical formulation comprises about 9 mg GDC-0077 (INAVOLISIB).

A specific embodiment of present invention relates to a pharmaceutical formulation as described herein, wherein the pharmaceutical formulation comprises 3 mg GDC-0077 (INAVOLISIB) and a first filler in the amount of 50 to 80 mg/100 mg core tablet weight.

A specific embodiment of present invention relates to a pharmaceutical formulation as described herein, wherein the pharmaceutical formulation comprises 3 mg GDC-0077 (INAVOLISIB) and a first filler in the amount of 60 to 80 mg/100 mg core tablet weight

A specific embodiment of present invention relates to a pharmaceutical formulation as described herein, wherein the pharmaceutical formulation comprises 3 mg GDC-0077 (INAVOLISIB) and a first filler in the amount of about 69 mg/100 mg core tablet weight.

A specific embodiment of present invention relates to a pharmaceutical formulation as described herein, wherein the first filler is present in a total amount of about 50-80 wt. %.

A specific embodiment of present invention relates to a pharmaceutical formulation as described herein, wherein the first filler is present in a total amount of about 60-80 wt. %.

A specific preferred embodiment of present invention relates to a pharmaceutical formulation as described herein, wherein the first filler is present in a total amount of about 60-75 wt. %.

A specific preferred embodiment of present invention relates to a pharmaceutical formulation as described herein, wherein the first filler is present in a total amount of about 69 wt. %.

A specific embodiment of present invention relates to a pharmaceutical formulation as described herein, wherein the first filler is microcrystalline cellulose.

A specific embodiment of present invention relates to a pharmaceutical formulation as described herein, wherein the microcrystalline cellulose is Avicel PH-102™. Any filler similar, equivalent or the same as the afore-mentioned microcrystalline cellulose can be used if they have similar properties, e.g. particle size distribution.

A specific embodiment of present invention relates to a pharmaceutical formulation as described herein, wherein the pharmaceutical formulation comprises 3 mg GDC-0077 (INAVOLISIB) and a second filler in the amount of 15 to 30 mg/100 mg core tablet weight.

A specific embodiment of present invention relates to a pharmaceutical formulation as described herein, wherein the pharmaceutical formulation comprises 3 mg GDC-0077 (INAVOLISIB) and a second filler in the amount of 20 to 30 mg/100 mg core tablet weight.

A specific embodiment of present invention relates to a pharmaceutical formulation as described herein, wherein the pharmaceutical formulation comprises 3 mg GDC-0077 (INAVOLISIB) and a second filler in the amount of about 23 mg/100 mg core tablet weight.

A specific embodiment of present invention relates to a pharmaceutical formulation as described herein, wherein the second filler is present in a total amount of about 15-30 wt. %, preferably about 20-30 wt. %, preferably 20-25 wt. %, for example about 23 wt. %.

A specific embodiment of present invention relates to a pharmaceutical formulation as described herein, wherein the second filler is spray dried lactose.

A specific embodiment of present invention relates to a pharmaceutical formulation as described herein, wherein the spray dried lactose is Fast Flo 316™. Any filler, equivalent or the same as the afore-mentioned spray dried lactose can be used if they have similar properties, e.g. flowability (Hausner ratio).

A specific embodiment of present invention relates to a pharmaceutical formulation as described herein, wherein the pharmaceutical formulation comprises 3 mg GDC-0077 (INAVOLISIB) and a lubricant in the amount of 0.5 to 2 mg/100 mg core tablet weight.

A specific embodiment of present invention relates to a pharmaceutical formulation as described herein, wherein the pharmaceutical formulation comprises 3 mg GDC-0077 (INAVOLISIB) and a lubricant in the amount of about 0.2 mg to about 1.5 mg/100 mg core tablet weight.

A specific embodiment of present invention relates to a pharmaceutical formulation as described herein, wherein the pharmaceutical formulation comprises 3 mg GDC-0077 (INAVOLISIB) and a lubricant in the amount of about 1 mg/100 mg core tablet weight.

A specific embodiment of present invention relates to a pharmaceutical formulation as described herein, wherein the lubricant is present in a total amount of about 0.2-5 wt. %, preferably about 0.2-2 wt. %, preferably 0.5-1.5 wt. %, for example about 1 wt. %.

A specific embodiment of present invention relates to a pharmaceutical formulation as described herein, wherein the lubricant is magnesium stearate.

A specific embodiment of present invention relates to a pharmaceutical formulation as described herein, wherein the magnesium stearate is MF-2-V™. Any lubricant similar, equivalent or the same as the afore-mentioned magnesium stearate can be used if they have similar properties, e.g. particle size distribution.

A specific embodiment of present invention relates to a pharmaceutical formulation as described herein, wherein the pharmaceutical formulation comprises 3 mg GDC-0077 (INAVOLISIB) and a disintegrant in the amount of 2 to 8 mg/100 mg core tablet weight.

A specific embodiment of present invention relates to a pharmaceutical formulation as described herein, wherein the pharmaceutical formulation comprises 3 mg GDC-0077 (INAVOLISIB) and a disintegrant in the amount of 3 to 6 mg/100 mg core tablet weight.

A specific embodiment of present invention relates to a pharmaceutical formulation as described herein, wherein the pharmaceutical formulation comprises 3 mg GDC-0077 (INAVOLISIB) and a disintegrant in the amount of about 4 mg/100 mg core tablet weight.

A specific embodiment of present invention relates to a pharmaceutical formulation as described herein, wherein the disintegrant is present in a total amount of about 1-10 wt. %, preferably about 2-7 wt. %, preferably 2-5 wt. %, for example about 4 wt. %.

A specific embodiment of present invention relates to a pharmaceutical formulation as described herein, wherein the disintegrant is sodium starch glycolate.

A specific embodiment of present invention relates to a pharmaceutical formulation as described herein, wherein the sodium starch glycolate is Primojel™. Any disintegrant similar, equivalent or the same as the afore-mentioned sodium starch glycolate can be used if they have similar properties, e.g. particle size distribution.

A specific embodiment of present invention relates to a pharmaceutical formulation as described herein, wherein the pharmaceutical formulation comprises 9 mg GDC-0077 (INAVOLISIB) and a first filler in the amount of 150 to 230 mg/300 mg core tablet weight.

A specific embodiment of present invention relates to a pharmaceutical formulation as described herein, wherein the pharmaceutical formulation comprises 9 mg GDC-0077 (INAVOLISIB) and a first filler in the amount of about 207 mg/300 mg core tablet weight.

A specific embodiment of present invention relates to a pharmaceutical formulation as described herein, wherein the pharmaceutical formulation comprises 9 mg GDC-0077 (INAVOLISIB) and a second filler in the amount of 50 to 80 mg/300 mg core tablet weight.

A specific embodiment of present invention relates to a pharmaceutical formulation as described herein, wherein the pharmaceutical formulation comprises 9 mg GDC-0077 (INAVOLISIB) and a second filler in the amount of about 69 mg/300 mg core tablet weight.

A specific embodiment of present invention relates to a pharmaceutical formulation as described herein, wherein the first filler is spray dried lactose.

A specific embodiment of present invention relates to a pharmaceutical formulation as described herein, wherein the spray dried lactose is Fast Flo 316™. Any filler similar, equivalent or the same as the afore-mentioned spray dried lactose can be used if they have similar properties, e.g. flowability (Hausner ratio).

A specific embodiment of present invention relates to a pharmaceutical formulation as described herein, wherein the pharmaceutical formulation comprises 9 mg GDC-0077 (INAVOLISIB) and a lubricant in the amount of 1 to 5 mg/300 mg core tablet weight.

A specific embodiment of present invention relates to a pharmaceutical formulation as described herein, wherein the pharmaceutical formulation comprises 9 mg GDC-0077 (INAVOLISIB) and a lubricant in the amount of about 2 mg to about 4 mg/300 mg core tablet weight.

A specific embodiment of present invention relates to a pharmaceutical formulation as described herein, wherein the pharmaceutical formulation comprises 9 mg GDC-0077 (INAVOLISIB) and a lubricant in the amount of about 3 mg/300 mg core tablet weight.

A specific embodiment of present invention relates to a pharmaceutical formulation as described herein, wherein the lubricant is magnesium stearate.

A specific embodiment of present invention relates to a pharmaceutical formulation as described herein, wherein the magnesium stearate is MF-2-V™. Any lubricant similar, equivalent or the same as the afore-mentioned magnesium stearate can be used if they have similar properties, e.g. particle size distribution.

A specific embodiment of present invention relates to a pharmaceutical formulation as described herein, wherein the pharmaceutical formulation comprises 3 mg GDC-0077 (INAVOLISIB) and a disintegrant in the amount of 9 to 15 mg/300 mg core tablet weight.

A specific embodiment of present invention relates to a pharmaceutical formulation as described herein, wherein the pharmaceutical formulation comprises 9 mg GDC-0077 (INAVOLISIB) and a disintegrant in the amount of 11 to 13 mg/300 mg core tablet weight.

A specific embodiment of present invention relates to a pharmaceutical formulation as described herein, wherein the pharmaceutical formulation comprises 9 mg GDC-0077 (INAVOLISIB) and a disintegrant in the amount of about 12 mg/100 mg core tablet weight.

A specific embodiment of present invention relates to a pharmaceutical formulation as described herein, wherein the disintegrant is sodium starch glycolate.

A specific embodiment of present invention relates to a pharmaceutical formulation as described herein, wherein the sodium starch glycolate is Primojel™. A specific embodiment of present invention relates to a pharmaceutical formulation as described herein, wherein the magnesium stearate is MF-2-V™. Any disintegrant similar, equivalent or the same as the afore-mentioned magnesium stearate can be used if they have similar properties, e.g. particle size distribution.

A specific embodiment of the present invention relates to a pharmaceutical formulation as described herein, wherein the core tablet weight is about 100 mg. A specific embodiment of the present invention relates to a pharmaceutical formulation as described herein, wherein the core tablet weight is about 300 mg.

A specific embodiment of the present invention relates to a pharmaceutical formulation as described herein, wherein the coated tablet weight is about 104 mg. A specific embodiment of the present invention relates to a pharmaceutical formulation as described herein, wherein the coated tablet weight is about 312 mg.

A specific embodiment of the present invention relates to a pharmaceutical formulation as described herein, wherein the coated tablet weight is about 104 mg and the formulation comprises about 3 mg GDC-0077 (INAVOLISIB).

A specific embodiment of the present invention relates to a pharmaceutical formulation as described herein, wherein the coated tablet weight is about 312 mg and the formulation comprises about 9 mg GDC-0077 (INAVOLISIB).

A specific embodiment of present invention relates to a pharmaceutical formulation as described herein that is shown in Table 1A.

TABLE 1A
Pharmaceutical Formulation

| | | Quantity per Unit | Quantity per Unit |
|---|---|---|---|
| | | Dose (3 mg) | Dose (9 mg) |
| Components | Function | (mg/tablet) | (mg/tablet) |
| Tablet Core | | | |
| GDC-0077 INAVOLISIB | Active (API) | 3.0 | 9.0 |
| Microcrystalline Cellulose | Filler | 69.0 | 207.0 |
| Spray dried Lactose | Filler | 23.0 | 69.0 |
| Sodium Starch Glycolate | Disintegrant | 4.0 | 12.0 |
| Magnesium Stearate | Lubricant | 1.0 | 3.0 |
| Weight of Tablet Core | | 100.0 | 300.0 |
| Tablet core dimensions | | 6 mm round bi- | 13 × 6.02 mm |
| | | convex, 3.4 mm thick | oval, 4.4 mm thick |
| Weight of Film-Coating | | 4.0 | 12.0 |
| Mixture | | | |
| Total Weight of Film-Coated | | 104.0 | 312.0 |
| Tablets | | | |

A specific embodiment of present invention relates to a pharmaceutical formulation as described herein, wherein the pharmaceutical formulation comprises an intragranular compartment and an extragranular compartment.

The pharmaceutical formulation of this invention, e.g. with an intragranular and an extragranular compartment, i.e., with intragranular and extragranular excipients, has the advantage to improve processability, prevention of processing issues, and an improved release profile. Together with other factors it also leads to an improved stability.

A specific embodiment of present invention relates to a pharmaceutical formulation as described herein wherein the pharmaceutical formulation comprises:
one or more intragranular excipients; and
one or more extragranular excipients.

A specific embodiment of present invention relates to a pharmaceutical formulation as described herein wherein the pharmaceutical formulation contains one, two, three, four or five intragranular excipients.

A specific embodiment of present invention relates to a pharmaceutical formulation as described herein wherein the pharmaceutical formulation contains one, two, three or four intragranular excipients.

A specific embodiment of present invention relates to a pharmaceutical formulation as described herein wherein the pharmaceutical formulation contains one, two, or three intragranular excipients.

A specific embodiment of present invention relates to a pharmaceutical formulation as described herein, wherein the intragranular compartment comprises GDC-0077 (INAVOLISIB) in the range of 2-12 wt. %.

A specific embodiment of present invention relates to a pharmaceutical formulation as described herein, wherein the intragranular compartment comprises GDC-0077 (INAVOLISIB) in the range of 2-5 wt. %.

A specific embodiment of present invention relates to a pharmaceutical formulation as described herein, wherein the intragranular compartment comprises GDC-0077 (INAVOLISIB) in an amount of about 3 wt. %.

A specific embodiment of present invention relates to a pharmaceutical formulation as described herein, wherein the intragranular compartment comprises GDC-0077 (INAVOLISIB) having a particle size distribution having D50 between 10 and 132 μm, and/or a D90 below 264 μm. Such a particle size distribution has the advantage to improve processability, prevention of processing issues, improved release profile, and an improved stability. A specific embodiment of present invention relates to a pharmaceutical formulation as described herein, wherein the intragranular compartment comprises a first filler in the range of 60-80 wt. %.

A specific embodiment of present invention relates to a pharmaceutical formulation as described herein, wherein the intragranular compartment comprises a first filler in the amount of about 69 wt. %.

A specific embodiment of present invention relates to a pharmaceutical formulation as described herein, wherein the first intragranular filler is microcrystalline cellulose as described herein.

A specific embodiment of present invention relates to a pharmaceutical formulation as described herein, wherein the intragranular compartment comprises a second filler in the range of 20-30 wt. %.

A specific embodiment of present invention relates to a pharmaceutical formulation as described herein, wherein the intragranular compartment comprises a second filler in the amount of about 23 wt. %.

A specific embodiment of present invention relates to a pharmaceutical formulation as described herein, wherein the second intragranular filler is lactose as described herein.

A specific embodiment of present invention relates to a pharmaceutical formulation as described herein, wherein the second intragranular filler is spray dried lactose as described herein.

A specific embodiment of present invention relates to a pharmaceutical formulation as described herein, wherein the intragranular compartment comprises a disintegrant in the range of 1-4 wt. %.

A specific embodiment of present invention relates to a pharmaceutical formulation as described herein, wherein the intragranular compartment comprises a disintegrant in the amount of about 2.0 wt. %.

A specific embodiment of present invention relates to a pharmaceutical formulation as described herein, wherein the intragranular disintegrant is sodium starch glycolate as described herein.

A specific embodiment of present invention relates to a pharmaceutical formulation as described herein, wherein the intragranular compartment comprises a lubricant in the range of 0.1-1.5 wt. %.

A specific embodiment of present invention relates to a pharmaceutical formulation as described herein, wherein the intragranular compartment comprises a lubricant in the amount of about 0.5 wt. %.

A specific embodiment of present invention relates to a pharmaceutical formulation as described herein, wherein the intragranular lubricant is magnesium stearate as described herein.

A specific embodiment of present invention relates to a pharmaceutical formulation as described herein, wherein the extragranular compartment comprises a disintegrant in the range of 1-4 wt. %.

A specific embodiment of present invention relates to a pharmaceutical formulation as described herein, wherein the extragranular compartment comprises a disintegrant in the amount of about 2.0 wt. %.

A specific embodiment of present invention relates to a pharmaceutical formulation as described herein, wherein the extragranular disintegrant is sodium starch glycolate as described herein.

A specific embodiment of present invention relates to a pharmaceutical formulation as described herein, wherein the extragranular compartment comprises a lubricant in the range of 0.1-1.5 wt. %.

A specific embodiment of present invention relates to a pharmaceutical formulation as described herein, wherein the extragranular compartment comprises a lubricant in the amount of about 0.5 wt. %.

A specific embodiment of present invention relates to a pharmaceutical formulation as described herein, wherein the extragranular lubricant is magnesium stearate as described herein.

A specific embodiment of present invention relates to a pharmaceutical formulation as described herein, wherein the pharmaceutical formulation is as shown in Table 2A. A specific embodiment of present invention relates to a pharmaceutical formulation as described herein, wherein the pharmaceutical formulation is as shown in Table 2B.

A specific embodiment of present invention relates to a pharmaceutical formulation as described herein, wherein the intragranular compartment comprises GDC-0077 (INAVOLISIB) in the range of 2-12 mg/100 mg core tablet weight.

A specific embodiment of present invention relates to a pharmaceutical formulation as described herein, wherein the intragranular compartment comprises GDC-0077 (INAVOLISIB) in the range of 2-5 mg/100 mg core tablet weight.

A specific embodiment of present invention relates to a pharmaceutical formulation as described herein, wherein the intragranular compartment comprises GDC-0077 (INAVOLISIB) in an amount of about 3 mg/100 mg core tablet weight.

A specific embodiment of present invention relates to a pharmaceutical formulation as described herein, wherein the intragranular compartment comprises a first filler in the range of 60-80 mg/100 mg core tablet weight.

A specific embodiment of present invention relates to a pharmaceutical formulation as described herein, wherein the intragranular compartment comprises a first filler in the amount of about 69 mg/100 mg core tablet weight.

A specific embodiment of present invention relates to a pharmaceutical formulation as described herein, wherein the first intragranular filler is microcrystalline cellulose as described herein.

A specific embodiment of present invention relates to a pharmaceutical formulation as described herein, wherein the intragranular compartment comprises a second filler in the range of 20-30 mg/100 mg core tablet weight.

A specific embodiment of present invention relates to a pharmaceutical formulation as described herein, wherein the intragranular compartment comprises a second filler in the amount of about 23 mg/100 mg core tablet weight.

A specific embodiment of present invention relates to a pharmaceutical formulation as described herein, wherein the second intragranular filler is spray dried lactose as described herein.

A specific embodiment of present invention relates to a pharmaceutical formulation as described herein, wherein the intragranular compartment comprises a disintegrant in the range of 1-4 mg/100 mg core tablet weight.

A specific embodiment of present invention relates to a pharmaceutical formulation as described herein, wherein the intragranular compartment comprises a disintegrant in the amount of about 2.0 mg/100 mg core tablet weight.

A specific embodiment of present invention relates to a pharmaceutical formulation as described herein, wherein the intragranular disintegrant is sodium starch glycolate as described herein.

A specific embodiment of present invention relates to a pharmaceutical formulation as described herein, wherein the intragranular compartment comprises a lubricant in the range of 0.1-1.5 mg/100 mg core tablet weight.

A specific embodiment of present invention relates to a pharmaceutical formulation as described herein, wherein the intragranular compartment comprises a lubricant in the amount of about 0.5 mg/100 mg core tablet weight.

A specific embodiment of present invention relates to a pharmaceutical formulation as described herein, wherein the intragranular lubricant is magnesium stearate as described herein.

A specific embodiment of present invention relates to a pharmaceutical formulation as described herein, wherein the extragranular compartment comprises a disintegrant in the range of 1-4 mg/100 mg core tablet weight.

A specific embodiment of present invention relates to a pharmaceutical formulation as described herein, wherein the extragranular compartment comprises a disintegrant in the amount of about 2.0 mg/100 mg core tablet weight.

A specific embodiment of present invention relates to a pharmaceutical formulation as described herein, wherein the extragranular disintegrant is sodium starch glycolate as described herein.

A specific embodiment of present invention relates to a pharmaceutical formulation as described herein, wherein the extragranular compartment comprises a lubricant in the range of 0.1-1.5 mg/100 mg core tablet weight.

A specific embodiment of present invention relates to a pharmaceutical formulation as described herein, wherein the extragranular compartment comprises a lubricant in the amount of about 0.5 mg/100 mg core tablet weight.

A specific embodiment of present invention relates to a pharmaceutical formulation as described herein, wherein the extragranular lubricant is magnesium stearate as described herein.

As used herein, the term “compartment” relates to a region of the formulation, e.g. an intragranular region or extragranular region. The term “compartment” does not necessarily imply a physical barrier of any sort; when used to refer to intragranular and extragranular regions, the separation between these is typically defined solely by the extent of the granule in the formulation, typically no further barrier is provided.

A specific embodiment of present invention relates to a pharmaceutical formulation as described herein, wherein the pharmaceutical formulation is as shown in Table 2A.

TABLE 2A
Pharmaceutical Formulation

| | | | 3 mg | 9 mg |
|---|---|---|---|---|
| | | | Amount | Amount |
| | | | per unit | per unit |
| Component | Function | % | (mg) | (mg) |
| Tablet Core | | | | |
| GDC-0077 | Active | 3.00 | 3.00 | 9.00 |
| INAVOLISIB | | | | |
| Microcrystalline | Filler | 69.00 | 69.00 | 207.00 |
| Cellulose | | | | |
| Spray Dried | Filler | 23.00 | 23.00 | 69.00 |
| Lactose | | | | |
| Sodium Starch | Disintegrant | 4.00 | 4.00 | 12.00 |
| Glycolate | | | | |
| Magnesium | Lubricant | 1.00 | 1.00 | 3.00 |
| Stearate | | | | |
| Weight core tablets | | 100.00 | 100.00 | 300.00 |
| Film Coat | | | | |
| Coating agent | Coating | 4.00 | 4.00 | 12.00 |
| | agent | | | |
| Purified Water | Vehicle | 0.50 | — | — |

| | | |
|---|---|---|
| Weight film-coated tablets | 104.00 | 312.00 |

A specific embodiment of present invention relates to a pharmaceutical formulation as described herein, wherein the pharmaceutical formulation is as shown in Table 2B.

TABLE 2B
Pharmaceutical Formulation
Formulation composition of Inavolisib 3 mg and 9 mg film-coated tablets

| | 3 mg | | 9 mg | |
|---|---|---|---|---|
| | Amount per | Amount per | Amount per | Amount per |
| Function | unit (mg) | unit (%) | unit (mg) | unit (%) |

Intragranular

| | | | | | |
|---|---|---|---|---|---|
| GDC-0077 | Active | 3.00 | 3.00 | 9.00 | 3.00 |
| INAVOLISIB | Ingredient | | | | |
| Microcrystalline | Filler | 20.00 | 20.00 | 50.00 | 16.67 |
| Cellulose, | | | | | |
| Part A^{a, b} | | | | | |
| Microcrystalline | Filler | 19.00 | 19.00 | 57.00 | 19.00 |
| Cellulose, Part B ^{b} | | | | | |
| Microcrystalline | Filler | 30.00 | 30.00 | 100.00 | 33.33 |
| Cellulose, Part C ^{b} | | | | | |
| Spray Dried | Filler | 23.00 | 23.00 | 69.00 | 23.00 |
| Lactose | | | | | |
| Sodium Starch | Disintegrant | 2.00 | 2.00 | 6.00 | 2.00 |
| Glycolate | | | | | |
| Magnesium | Lubricant | 0.50 | 0.50 | 1.50 | 0.50 |
| Stearate | | | | | |

Extragranular

| | | | | | |
|---|---|---|---|---|---|
| Sodium Starch | Disintegrant | 2.00 | 2.00 | 6.00 | 2.00 |
| Glycolate | | | | | |
| Magnesium | Lubricant | 0.50 | 0.50 | 1.50 | 0.50 |
| Stearate | | | | | |
| Weight core | | 100.00 | 100.00 | 300.00 | 100.00 |
| tablets | | | | | |
| Coating agent | Coating agent | 4.00 | +4% weight | 12.00 | +4% weight |
| | | | gain | | gain |
| Purified Water ^{c} | Vehicle | 22.67 | q.s. | 68.00 | q.s |

| | | | | |
|---|---|---|---|---|
| Weight film coated tablets | 104.00 | — | 312.00 | — |

^{a}The amount of API and MCC will be defined in accordance to the content of the API based on the CoA
^{b} The total amount of intragranular MCC in % is identical for 3 mg and 9 mg (i.e. 69.00%)
^{c} Removed during coating. The coating suspension is prepared at 15% w/w solids content. To be prepared in excess to allow proper functionality of the coating suspension system.

A specific embodiment of present invention relates to a pharmaceutical formulation as described herein, wherein the pharmaceutical formulation is as shown in Table 2C.

TABLE 2C
Pharmaceutical Formulation
Formulation composition of Inavolisib 3 mg and 9 mg film-coated tablets

| | | | 3 mg | 9 mg |
|---|---|---|---|---|
| | | | Amount | Amount |
| | | | per unit | per unit |
| Component | Function | % | (mg) | (mg) |
| Intragranular | | | | |
| GDC-0077 | Active | 3.00 | 3.00 | 9.00 |
| INAVOLISIB | ingredient | | | |
| Microcrystalline | Filler | 69.00 | 69.00 | 207.00 |
| Cellulose | | | | |
| Spray Dried | Filler | 23.00 | 23.00 | 69.00 |
| Lactose | | | | |
| Sodium Starch | Disintegrant | 2.00 | 2.00 | 6.00 |
| Glycolate | | | | |
| Magnesium | Lubricant | 0.50 | 0.50 | 1.50 |
| Stearate | | | | |
| Extragranular | | | | |
| Sodium Starch | Disintegrant | 2.00 | 2.00 | 6.00 |
| Glycolate | | | | |
| Magnesium | Lubricant | 0.50 | 0.50 | 1.50 |
| Stearate | | | | |
| Weight core tablets | | 100.00 | 100.00 | 300.00 |
| Coating agent | Coating | +4% | 4.00 | 12.00 |
| | agent | weight | | |
| | | gain | | |
| Purified Water | Vehicle | q.s | 22.67 | 68.00 |

| | | |
|---|---|---|
| Weight film-coated tablets | 104.00 | 312.00 |

A specific embodiment of present invention relates to a pharmaceutical formulation as described herein that is shown in Table 3.

TABLE 3
Pharmaceutical Formulation

| | Quantity per Unit Dose |
|---|---|
| Components | % w/w |
| Intra-Granular | |
| GDC-0077 | 3.0 |
| (INAVOLISIB) | |
| Microcrystalline | 69.0 |
| Cellulose | |
| Spray Dried Lactose | 23.0 |
| Sodium Starch | 2.0 |
| Glycolate | |
| Magnesium Stearate | 0.5 |
| Extra-Granular | |
| Sodium Starch | 2.0 |
| Glycolate | |
| Magnesium Stearate | 0.5 |
| Sub-Total (Core | 100 |
| Tablet) | |
| Colored film coating | 4.0 |

A further aspect of the present invention relates to a pharmaceutical formulation as described herein, obtainable by a process comprising the steps of:
(i) blending GDC-0077 (INAVOLISIB) with one or more intragranular excipients;
(ii) dry granulating the blend resulting from step (i);
(iii) blending the granulated product of step (ii) with one or more extragranular excipients;
(iv) tableting the blend resulting from step (iii) into tablets; and
(v) applying a colored coating formulation to the exterior of the tablets.

In preferred aspects of such a process, the particles of the GDC-0077 (INAVOLISIB) in step (i) have a D50 value between 10 and 132 μm and D90 value below 264 μm.

A specific embodiment of the present invention relates to such a formulation obtainable by the above-described process, wherein process step (i) is performed as a sandwiching step with multiple alternating layers of one or more intragranular excipients and GDC-0077 (INAVOLISIB) being added to the blender before blending. The different layers may be the same or different with regard to thickness/amount of GDC-0077 (INAVOLISIB). Such a process offers improved processing and more thorough mixing of the components. In addition, sandwiching ensures a homogeneous mixture (blend uniformity).

The distribution of intragranular microcrystalline cellulose parts A, B and C may differ for different embodiments, in particular the distribution may be:
part A: about 29.0%
part B: about 27.5%
part C: about 43.5%
(based on the total amount of intragranular microcrystalline cellulose).

The distribution of intragranular microcrystalline cellulose parts A, B and C may differ for different embodiments, in particular the distribution may be:
part A: about 24.2%
part B: about 27.5%
part C: about 48.3%
(based on the total amount of intragranular microcrystalline cellulose)

A specific embodiment of present invention relates to a pharmaceutical formulation, wherein for the intragranular excipients microcrystalline cellulose as filler may be added by the sandwiching method described herein with one, two or three layers (parts A, B, C) with interleaving layers of GDC-0077 (INAVOLISIB).

In a preferred embodiment of two layers (parts A, B) of microcrystalline cellulose are used with one interleaving layer of GDC-0077 (INAVOLISIB).

In a preferred embodiment of two layers (parts A, B, C) of microcrystalline cellulose are used with one interleaving layer of GDC-0077 (INAVOLISIB).

A specific embodiment of the present invention relates to a pharmaceutical formulation as described herein, wherein the process step (i) is performed as a sandwiching step with multiple alternating layers of microcrystalline cellulose and GDC-0077 (INAVOLISIB), wherein there is a first layer of microcrystalline cellulose (part A), a layer of GDC-0077 (INAVOLISIB), and a second layer of microcrystalline cellulose (part B).

A specific embodiment of the present invention relates to a pharmaceutical formulation as described herein, wherein the process step (i) is performed as a sandwiching step with multiple alternating layers of microcrystalline cellulose and GDC-0077 (INAVOLISIB), wherein there is a first layer of microcrystalline cellulose (part A), a layer of GDC-0077 (INAVOLISIB), a second layer of microcrystalline cellulose (part B), and a third layer of microcrystalline cellulose (part C). In a preferred embodiment, a third layer of microcrystalline cellulose (part C) is added after a blending step together with other intragranular excipients.

The different layers may be the same or different with regard to thickness/amount of GDC-0077 (INAVOLISIB).

The different layers may be the same or different with regard to distribution of microcrystalline cellulose between the different layers.

A specific embodiment of present invention relates to a pharmaceutical formulation, wherein the sandwiching method comprises the steps:
(i) adding microcrystalline cellulose part A to the blender;
(ii) adding GDC-0077 (INAVOLISIB) to the blender;
(iii) adding microcrystalline cellulose part B to the blender;
(iv) blending; and
(v) sieving.

A specific embodiment of present invention relates to a pharmaceutical formulation, wherein the sandwiching method comprises the steps:
(i) adding microcrystalline cellulose part A to the blender;
(ii) adding GDC-0077 (INAVOLISIB) to the blender;
(iii) adding microcrystalline cellulose part B to the blender;
(iv) blending;
(v) sieving; and
(vi) adding microcrystalline cellulose part C to the blender.

A specific embodiment of present invention relates to a pharmaceutical formulation, wherein the process comprises the steps:
(i) adding a first filler (part A) to the blender;
(ii) adding GDC-0077 (INAVOLISIB) to the blender;
(iii) adding a first filler part B to the blender;
(iv) blending;
(v) sieving;
(vi) adding a first filler part C to the blender;
(vii) adding a second filler;
(vii) adding a disintegrant
(viii) sieving, and
(ix) blending.

In certain embodiments, the microcrystalline cellulose part C is added together with sodium starch glycolate and lactose.

The different layers may be the same or different with regard to thickness/amount of GDC-0077 (INAVOLISIB).

A further aspect of the present invention relates to a pharmaceutical formulation as described herein, obtainable by a process comprising the steps of:
(i) Adding the microcrystalline cellulose (part A and B) and GDC-0077 (INAVOLISIB) to a blender and blending;
(ii) Passing the blend from Step (i), lactose, and the intragranular portion of sodium starch glycolate, through a screening mill into a second bin blender;
(iii) Blending the powder blend from Step (ii);
(iv) Adding the intragranular portion of magnesium stearate and blending;
(v) Dry granulating the blend (from Step (iv)) using a roller compactor and passing through a screening mill;
(vi) Transferring the granules (from Step (v)) to a blender, adding the extra granular portion of sodium starch glycolate, and blending;
(vii) Adding the extragranular portion of magnesium stearate and blending;
(viii) Compressing the final blend (from Step (vii)) into tablets by using a tablet press; and
(ix) Preparing a film-coating suspension using the colored film-coating and purified water; and
(x) Spraying the coating suspension onto the tablet cores using a pan coater.

A further aspect of the present invention relates to a pharmaceutical formulation as described herein, obtainable by a process comprising the steps of:
(i) Adding the microcrystalline cellulose (part A and) and GDC-0077 (INAVOLISIB) to a bin blender and blending;
(ii) Passing the blend from Step (i), microcrystalline cellulose (part C), lactose, and the intragranular portion of sodium starch glycolate, through a screening mill into a second bin blender;
(iii) Blending the powder blend from Step (ii);
(iv) Adding the intragranular portion of magnesium stearate and blending;
(v) Dry granulating the blend (from Step (iv)) using a roller compactor and passing through a screening mill;
(vi) Transferring the granules (from Step (v)) to a blender, adding the extra granular portion of sodium starch glycolate, and blending;
(vii) Adding the extragranular portion of magnesium stearate and blending;
(viii) Compressing the final blend (from Step (vii)) into tablets by using a tablet press;
and
(ix) Preparing a film-coating suspension using the colored film-coating and purified water; and
(x) Spraying the coating suspension onto the tablet cores using a pan coater.

A specific embodiment of present invention relates to a pharmaceutical formulation, wherein the process comprises handsieving of intra- and extragranular magnesium stearate and of extragranular sodium starch glycolate prior addition.

In this application, blender in particular preferably means bin blender.

In certain embodiments, oxidizing tableting excipients, e.g. copovidone, should be avoided to avoid degradation of the GDC-0077 (INAVOLISIB).

In certain embodiments, wet processing of the GDC-0077 (INAVOLISIB) should be avoided to avoid degradation of the GDC-0077 (INAVOLISIB).

A specific embodiment of present invention relates to the use of a pharmaceutical formulation as described herein, for the treatment of cancer.

A specific embodiment of present invention relates to the use of a pharmaceutical formulation as described above, for the treatment of breast cancer.

A specific embodiment of present invention relates to the use of a pharmaceutical formulation as described above, wherein the patient has PIK3CA mutant, hormone receptor-positive, Her2-negative, locally advanced, or metastatic breast cancer.

A specific embodiment of present invention relates to the use of a pharmaceutical formulation as described above, for the treatment of cancers associated with PIK3CA mutations.

A specific embodiment of present invention relates to the use of a pharmaceutical formulation as described above, for the treatment of cancers associated with PIK3CA mutations, wherein the patients have mutant PIK3CA having mutations at one or more of positions 88, 106, 111, 118, 345, 420, 453, 542, 545, 546, 1043, 1047, and 1049.

A specific embodiment of present invention relates to the use of a pharmaceutical formulation as described above, for the treatment of cancers associated with PIK3CA mutations, wherein the patient has mutant PIK3CA having mutations at one or more of H1047, E545, E542, Q546, N345, C420, M1043, G1049, E453, K111, G106, G118, and R88.

A specific embodiment of present invention relates to the use of a pharmaceutical formulation as described above, for the treatment of cancers associated with PIK3CA mutations, wherein the patient has mutant PIK3CA containing one or more mutations selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to the use of a pharmaceutical formulation as described above, for the treatment of cancers associated with PIK3CA mutations, wherein the patient has mutant PIK3CA containing one or more mutations selected from the group consisting of E542K, E545K, Q546R, H1047L, and H1047R.

A specific embodiment of present invention relates to the use of a pharmaceutical formulation as described above, for the treatment of cancers associated with PIK3CA mutations, wherein the patient has breast cancer expressing a PIK3CA mutant selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to the use of a pharmaceutical formulation as described above, for the treatment of cancers associated with PIK3CA mutations, wherein the patient has breast cancer expressing a PIK3CA mutant selected from the group consisting of E542K, E545K, Q546R, H1047L, and H1047R.

A specific embodiment of present invention relates to the use of a pharmaceutical formulation as described herein, in a method of treatment of cancer.

A specific embodiment of present invention relates to the use of a pharmaceutical formulation as described herein, in a method of treatment of breast cancer.

A specific embodiment of present invention relates to the use of a pharmaceutical formulation in a method of treatment as described herein, wherein the patient has PIK3CA mutant, hormone receptor-positive, Her2-negative, locally advanced, or metastatic breast cancer.

A specific embodiment of present invention relates to the use of a pharmaceutical formulation in a method of treatment as described herein, for the treatment of cancers associated with PIK3CA mutations.

A specific embodiment of present invention relates to the use of a pharmaceutical formulation in a method of treatment as described herein, for the treatment of cancers associated with PIK3CA mutations, wherein the patients have mutant PIK3CA having mutations at one or more of positions 88, 106, 111, 118, 345, 420, 453, 542, 545, 546, 1043, 1047, and 1049.

A specific embodiment of present invention relates to the use of a pharmaceutical formulation in a method of treatment as described herein, for the treatment of cancers associated with PIK3CA mutations, wherein the patient has mutant PIK3CA having mutations at one or more of H1047, E545, E542, Q546, N345, C420, M1043, G1049, E453, K111, G106, G118, and R88.

A specific embodiment of present invention relates to the use of a pharmaceutical formulation in a method of treatment as described herein, for the treatment of cancers associated with PIK3CA mutations, wherein the patient has mutant PIK3CA containing one or more mutations selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to the use of a pharmaceutical formulation in a method of treatment as described herein, for the treatment of cancers associated with PIK3CA mutations, wherein the patient has mutant PIK3CA containing one or more mutations selected from the group consisting of E542K, E545K, Q546R, H1047L, and H1047R.

A specific embodiment of present invention relates to the use of a pharmaceutical formulation in a method of treatment as described herein, for the treatment of cancers associated with PIK3CA mutations, wherein the patient has breast cancer expressing a PIK3CA mutant selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to the use of a pharmaceutical formulation in a method of treatment as described herein, for the treatment of cancers associated with PIK3CA mutations, wherein the patient has breast cancer expressing a PIK3CA mutant selected from the group consisting of E542K, E545K, Q546R, H1047L, and H1047R.

A specific embodiment of present invention relates to the use of a pharmaceutical formulation as described herein, in the manufacture of a medicament for the treatment of cancer.

A specific embodiment of present invention relates to the use of a pharmaceutical formulation in the manufacture of a medicament for the treatment of cancer as described herein, wherein the patient has PIK3CA mutant, hormone receptor-positive, Her2-negative, locally advanced, or metastatic breast cancer.

A specific embodiment of present invention relates to the use of a pharmaceutical formulation in the manufacture of a medicament for the treatment of cancer as described herein, wherein the cancers are associated with PIK3CA mutations.

A specific embodiment of present invention relates to the use of a pharmaceutical formulation in the manufacture of a medicament for the treatment of cancers associated with PIK3CA mutations, wherein the patients have mutant PIK3CA having mutations at one or more of positions 88, 106, 111, 118, 345, 420, 453, 542, 545, 546, 1043, 1047, and 1049.

A specific embodiment of present invention relates to the use of a pharmaceutical formulation in the manufacture of a medicament for the treatment of cancers associated with PIK3CA mutations, wherein the patient has mutant PIK3CA having mutations at one or more of H1047, E545, E542, Q546, N345, C420, M1043, G1049, E453, K111, G106, G118, and R88.

A specific embodiment of present invention relates to the use of a pharmaceutical formulation in the manufacture of a medicament for the treatment of cancers associated with PIK3CA mutations, wherein the patient has mutant PIK3CA containing one or more mutations selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to the use of a pharmaceutical formulation in the manufacture of a medicament for the treatment of cancers associated with PIK3CA mutations, wherein the patient has mutant PIK3CA containing one or more mutations selected from the group consisting of E542K, E545K, Q546R, H1047L, and H1047R.

A specific embodiment of present invention relates to the use of a pharmaceutical formulation in the manufacture of a medicament for the treatment of cancers associated with PIK3CA mutations, wherein the patient has breast cancer expressing a PIK3CA mutant selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to the use of a pharmaceutical formulation for the manufacture of a medicament for the treatment of cancers associated with PIK3CA mutations, wherein the patient has breast cancer expressing a PIK3CA mutant selected from the group consisting of E542K, E545K, Q546R, H1047L, and H1047R.

A specific embodiment of present invention relates to a pharmaceutical formulation as described herein, for use in the treatment of cancer.

A specific embodiment of present invention relates to the use of a pharmaceutical formulation as described herein, in the manufacture of a medicament for the treatment of cancer.

A specific embodiment of present invention relates to a pharmaceutical formulation use in the treatment of cancer as described herein, wherein the patient has PIK3CA mutant, hormone receptor-positive, Her2-negative, locally advanced, or metastatic breast cancer.

A specific embodiment of present invention relates to a pharmaceutical formulation for use in the treatment of cancer as described herein, wherein the cancers are associated with PIK3CA mutations.

A specific embodiment of present invention relates to a pharmaceutical formulation for use in the treatment of cancers associated with PIK3CA mutations, wherein the patients have mutant PIK3CA having mutations at one or more of positions 88, 106, 111, 118, 345, 420, 453, 542, 545, 546, 1043, 1047, and 1049.

A specific embodiment of present invention relates to a pharmaceutical formulation for use in the treatment of cancers associated with PIK3CA mutations, wherein the patient has mutant PIK3CA having mutations at one or more of H1047, E545, E542, Q546, N345, C420, M1043, G1049, E453, K111, G106, G118, and R88.

A specific embodiment of present invention relates to a pharmaceutical formulation for use in the treatment of cancers associated with PIK3CA mutations, wherein the patient has mutant PIK3CA containing one or more mutations selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to a pharmaceutical formulation for use in the treatment of cancers associated with PIK3CA mutations, wherein the patient has mutant PIK3CA containing one or more mutations selected from the group consisting of E542K, E545K, Q546R, H1047L and H1047R.

A specific embodiment of present invention relates to a pharmaceutical formulation for use in the treatment of cancers associated with PIK3CA mutations, wherein the patient has breast cancer expressing a PIK3CA mutant selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/T/V, G1049A/C/D/R/S, E453A/D/G/K/Q/V, K111N/R/E, G106A/D/R/S/V, G118D, and R88Q.

A specific embodiment of present invention relates to a pharmaceutical formulation for use in the treatment of cancers associated with PIK3CA mutations, wherein the patient has breast cancer expressing a PIK3CA mutant selected from the group consisting of E542K, E545K, Q546R, H1047L and H1047R.

In other specific embodiments, the cancer is selected from the group consisting of breast cancer, ovary cancer, cervix cancer, prostate cancer, testis cancer, genitourinary tract cancer, esophagus cancer, larynx cancer, glioblastoma, neuroblastoma, stomach cancer, skin cancer, keratoacanthoma, lung cancer, epidermoid carcinoma, large cell carcinoma, non-small cell lung carcinoma (NSCLC), small cell carcinoma, lung adenocarcinoma, bone cancer, colon cancer, adenoma, adenocarcinoma, thyroid cancer, follicular carcinoma, undifferentiated carcinoma, papillary carcinoma, seminoma, melanoma, sarcoma, bladder carcinoma, liver carcinoma, kidney carcinoma, pancreatic cancer, lymphoma, hairy cell leukemia, buccal cavity cancer, naso-pharyngeal cancer, pharynx cancer, lip cancer, tongue cancer, mouth cancer, small intestine cancer, colorectal cancer, large intestine cancer, rectal cancer, brain cancer, central nervous system caner, Hodgkin's lymphoma, leukemia, bronchus caner, thyroid cancer, liver cancer, intrahepatic bile duct cancer, hepatocellular cancer, gastric cancer, glioma, glioblastoma, endometrial cancer, kidney cancer, renal cancer, pelvis cancer, urinary bladder cancer, uterine corpus cancer, uterine cervix cancer, multiple myeloma, acute myelogenous leukemia, chronic myelogenous leukemia, lymphocytic leukemia, chronic lymphoid leukemia (CLL), myeloid leukemia, oral cavity cancer, non-Hodgkin lymphoma, melanoma, and villous colon adenoma.

In other specific embodiments, the cancer is selected from the group consisting of colon cancer, breast cancer, cervix cancer, stomach cancer, lung cancer, and multiple myeloma. In other specific embodiments, the cancer expresses a PIK3CA mutant.

In other specific embodiments, cancer expresses a PIK3CA mutant selected from the group consisting of E542K, E545K, Q546R, H1047L and H1047R.

In other specific embodiments, the cancer expresses a PTEN mutant.

In another specific embodiment, the invention relates to a pharmaceutical/formulation as described herein for use in a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising:
(i) GDC-0077 (INAVOLISIB), e.g. a formulation as defined herein;
(ii) palbociclib; and
(iii) fulvestrant,
wherein said combination therapy is administered over a 28-day cycle.

In another specific embodiment, the invention relates to a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising:
(i) GDC-0077 (INAVOLISIB), e.g. a formulation as defined herein;
(ii) palbociclib; and
(iii) fulvestrant,
wherein said combination therapy is administered over a 28-day cycle.

In another specific embodiment, the invention relates to a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering GDC-0077 (INAVOLISIB), e.g. a formulation as defined herein, QD on days 1-28 of a first 28-day cycle;
b. administering palbociclib QD on days 1-21 of a first 28-day cycle; and
c. administering fulvestrant on days 1 and 15 of a first 28-day cycle.

In another specific embodiment, the invention relates to a method as described above, further comprising one or more additional 28-day cycles comprising:
a. administering GDC-0077 (INAVOLISIB), e.g. a formulation as defined herein, on days 1-28 of each additional 28-day cycle;
b. administering palbociclib on days 1-21 of each additional 28-day cycle; and
c. administering fulvestrant on day 1 of each additional 28-day cycle.

In another specific embodiment, the invention relates to a method as described above, wherein GDC-0077 (INAVOLISIB) is administered at an amount of 9 mg.

In another specific embodiment, GDC-0077 (INAVOLISIB) is administered at an amount of 9 mg in an oral tablet.

In another specific embodiment, palbociclib is administered at an amount of 125 mg in an oral capsule or tablet.

In another specific embodiment, fulvestrant is administered at an amount of 500 mg by intramuscular (IM) infusion.

In another specific embodiment, this invention relates to a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising:
(i) GDC-0077 (INAVOLISIB), e.g. a formulation as defined herein;
(ii) palbociclib; and
(iii) letrozole,
wherein said combination therapy is administered over a 28-day cycle.

A specific embodiment of this invention relates to a method of treating hormone receptor positive and HER2 negative locally advanced or metastatic breast cancer in a patient having PIK3CA mutant, hormone receptor positive and HER2 negative locally advanced or metastatic breast cancer, the method comprising administering to the patient a combination therapy comprising a dosing regimen comprising:
a. administering GDC-0077 (INAVOLISIB), e.g. a formulation as defined herein, QD on days 1-28 of a first 28-day cycle;
b. administering palbociclib QD on days 1-21 of a first 28-day cycle; and
c. administering letrozole QD on days 1-28 of a first 28-day cycle.

Another specific embodiment of this invention comprises one or more additional 28-day cycles comprising:
a. administering GDC-0077 (INAVOLISIB), e.g. a formulation as defined herein, on days 1-28 of each additional 28-day cycle;
b. administering palbociclib on days 1-21 of each additional 28-day cycle; and
c. administering letrozole on days 1-28 of each additional 28-day cycle.

In another specific embodiment, GDC-0077 (INAVOLISIB) is administered at an amount of 3, 6, or 9 mg.

In another specific embodiment, GDC-0077 (INAVOLISIB) is administered at an amount of 9 mg.

In another specific embodiment, GDC-0077 (INAVOLISIB) is administered at an amount of 9 mg in an oral tablet, e.g. as defined herein.

In another specific embodiment, palbociclib is administered at an amount of 125 mg in an oral capsule or tablet.

In another specific embodiment, letrozole is administered at an amount of 2.5 mg in an oral tablet.

Another specific embodiment relates to a method of inhibiting tumor growth or producing/increasing tumor regression in a patient having PIK3CA mutant, hormone receptor positive and HER2 negative locally advanced or metastatic breast cancer, the method comprising administering to the patient a combination therapy according to the methods described herein.

In another specific embodiment, the patient has locally advanced or metastatic breast cancer not amenable to curative therapy.

In another specific embodiment, the patient has progression of disease during adjuvant endocrine treatment or within 12 months of completing adjuvant endocrine therapy with an aromatase inhibitor or tamoxifen.

In another specific embodiment, the patient has adequate hematologic and organ function within 14 days prior to initiation of study treatment.

In another specific embodiment, the patient is post-menopausal.

Another specific embodiment of this invention relates to a method of preventing or delaying development of resistance of a breast cancer to a therapy containing palbociclib, said method comprising administering a combination therapy comprising GDC-0077 (INAVOLISIB), Palbociclib, and fulvestrant, or a combination therapy comprises GDC-0077 (INAVOLISIB), Palbociclib, and letrozole, according to the method described herein.

### Embodiments

E1. The invention provides a pharmaceutical formulation in form of solid dosage form comprising GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the formula:

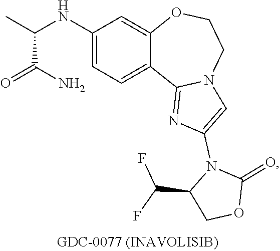 [SMILES: C[C@H](Nc1ccc2c(c1)OCCn1cc(N3C(=O)OC[C@H]3C(F)F)nc1-2)C(N)=O]

or a pharmaceutically acceptable salt thereof, wherein the pharmaceutical formulation comprises (i) a therapeutically effective amount of GDC-0077 (INAVOLISIB) or a pharmaceutically acceptable salt thereof, and (ii) a colored coating.

E2. The pharmaceutical formulation according to embodiment E1, wherein the solid dosage form is an immediate release tablet for oral administration.

E3. The pharmaceutical formulation according to embodiments E1 or E2, wherein the pharmaceutical formulation comprises (iii) one or more intragranular excipients, and (iv) one or more extragranular excipients.

E4. The pharmaceutical formulation according to embodiment E3, wherein the intragranular excipients are selected from the group consisting of binders, disintegrants, glidants, fillers, and lubricants.

E5. The pharmaceutical formulation according to any one of embodiments E3-E4, wherein the intragranular excipients are selected from the group consisting of fillers, disintegrants, and lubricants.

E6. A pharmaceutical formulation according to any one of embodiments E3-E5, wherein the extragranular excipients are selected from of binders, disintegrants, glidants, fillers, and lubricants.

E7. A pharmaceutical formulation according to embodiment E6, where the extragranular excipients are selected from the group consisting of disintegrants and lubricants. E8. A pharmaceutical formulation according to any one of embodiments E3-E7, wherein there are two intragranular fillers which are microcrystalline cellulose and spray dried lactose.

E9. A pharmaceutical formulation according to any one of embodiments E3-E8, wherein there is one intragranular disintegrant which is sodium starch glycolate.

E10. A pharmaceutical formulation according to any one of embodiments E3-E9, wherein there is one intragranular lubricant which is magnesium stearate.

E11. A pharmaceutical formulation according to any one of embodiments E3-E10, wherein there is one extragranular disintegrant which is sodium starch glycolate.

E12. A pharmaceutical formulation according to any one of embodiments E3-E11, wherein there is one extragranular lubricant which is magnesium stearate.

E13. A pharmaceutical formulation according to any one of embodiments E1-E12, wherein the colored coating is a water-soluble film coating.

E14. A pharmaceutical formulation according to embodiment E13, wherein the colored coating comprises one or more components selected from the group consisting of polymers, plasticizers, metal oxides. colored pigments, and any combination thereof.

E15. A pharmaceutical formulation according to embodiment E14, wherein the colored coating comprises one or more components selected from the group consisting of polyvinylalcohol (partially hydrolyzed), titanium dioxide, polyethylene glycol, talc, colored pigments, and any combination thereof.

E16. A pharmaceutical formulation according embodiment E14 or E15, wherein the colored pigments comprise one or more iron oxide(s).

E17. A pharmaceutical formulation according to embodiment E16, where the one or more iron oxide(s) are selected from the group consisting of iron oxide (red), iron oxide (yellow), iron oxide (black).

E18. A pharmaceutical formulation according to embodiment E17, where the one or more iron oxide is iron oxide (red).

E19. A pharmaceutical formulation according to embodiment E17, where the one or more iron oxide is a combination of iron oxide (red) and iron oxide (yellow).

E20. A pharmaceutical formulation according to any one of embodiments E13-E19, wherein the colored coating consists of:

| | |
|---|---|
| Polyvinylalcohol (partially hydrolyzed) | about 40.0% |
| Titanium dioxide | about 10.0% |
| Macrogol/Polyethylene glycol | about 20.2% |
| Talc | about 14.8% |
| Iron oxide (red) | about 15.0% |

or

| | |
|---|---|
| Polyvinylalcohol (partially hydrolyzed) | about 40.0% |
| Titanium dioxide | about 22.1% |
| Macrogol/Polyethylene glycol | about 20.2% |
| Talc | about 14.8% |
| Iron oxide (red) | about 1.5% |
| Iron oxide (yellow) | about 1.4% |

or

| | |
|---|---|
| Polyvinylalcohol (partially hydrolyzed) | about 40.0% |
| Titanium dioxide | about 20.8% |
| Macrogol/Polyethylene glycol | about 20.2% |
| Talc | about 14.8% |
| Iron oxide (red) | about 0.8% |
| Iron oxide (yellow) | about 3.3% |
| Iron oxide (black) | about 0.1% |

E21. A pharmaceutical formulation according to embodiment E20, wherein the colored coating consists of:

| | |
|---|---|
| Polyvinylalcohol (partially hydrolyzed) | about 40.0% |
| Titanium dioxide | about 10.0% |
| Macrogol/Polyethylene glycol | about 20.2% |
| Talc | about 14.8% |
| Iron oxide (red) | about 15.0% |

E22. A pharmaceutical formulation according to embodiment E20, wherein the colored coating consists of:

| | |
|---|---|
| Polyvinylalcohol (partially hydrolyzed) | about 40.0% |
| Titanium dioxide | about 22.1% |
| Macrogol/Polyethylene glycol | about 20.2% |
| Talc | about 14.8% |
| Iron oxide (red) | about 1.5% |
| Iron oxide (yellow) | about 1.4% |

E23. A pharmaceutical formulation according to any one of embodiments E13-E22, wherein the colored coating is Opadry II™.

E24. A pharmaceutical formulation according to any one of embodiments E13-E23, wherein the weight of the film-coating mixture per tablet is in the range of about 3 mg to about 15 mg.

E25. A pharmaceutical formulation according to any one of embodiments E13-E24, wherein the weight of the film-coating mixture per tablet is about 4 mg or about 12 mg.

E26. A pharmaceutical formulation according to any one of embodiments E3-E25, that consists of:

| | Quantity per Unit Dose |
|---|---|
| Components | % w/w |
| Intra-Granular | |
| GDC-0077 | 3.0 |
| (INAVOLISIB) | |
| Microcrystalline | 69.0 |
| Cellulose | |
| Spray Dried Lactose | 23.0 |
| Sodium Starch | 2.0 |
| Glycolate | |
| Magnesium Stearate | 0.5 |
| Extra-Granular | |
| Sodium Starch | 2.0 |
| Glycolate | |
| Magnesium Stearate | 0.5 |
| Sub-Total (Core | 100 |
| Tablet) | |
| Colored film coating | 4.0 |

E27. A pharmaceutical formulation according to any one of embodiments E1-E26, for use in the treatment of a cancer.

E28. A pharmaceutical formulation according to embodiment E27 for the treatment of a cancer associated with PIK3CA mutations.

E29. A pharmaceutical formulation according to embodiment E27 or E28, for use in the treatment of breast cancer.

E30. A pharmaceutical formulation according to embodiment E29, for use in the treatment of breast cancer, wherein the breast cancer is ½L PIK3CAmt HR+ mBC.

E31. A pharmaceutical formulation according to any one of embodiments E1-E26, obtainable by the process comprising the steps of:
(i) blending GDC-0077 (INAVOLISIB) with one or more intragranular excipients;
(ii) dry granulating the blend resulting from step (i);
(iii) blending the granulated product of step (ii) with one or more extragranular excipients;
(iv) tableting the blend resulting from step (iii) into tablets; and
(v) applying a colored coating formulation to the exterior of the tablets.

E.32. A pharmaceutical formulation according to embodiment E31, wherein the particles of the GDC-0077 (INAVOLISIB) in step (i) have a D50 value between 10 and 132 μm and D90 value below 264 μm.

E33. A pharmaceutical formulation according to any one of embodiments E1-E26, obtainable by the process comprising the steps of:
(i) Adding the microcrystalline cellulose (part A and B) and GDC-0077 (INAVOLISIB) to a blender and blending;
(ii) Passing the blend from Step (i), lactose, and the intragranular portion of sodium starch glycolate, through a screening mill into a second blender;
(iii) Blending the powder blend from Step (ii);
(iv) Adding the intragranular portion of magnesium stearate and blending;
(v) Dry granulating the blend (from Step (iv)) using a roller compactor and passing through a screening mill;
(vi) Transferring the granules (from Step (v)) to a blender, adding the extra granular portion of sodium starch glycolate, and blending;
(vii) Adding the extragranular portion of magnesium stearate and blending;
(viii) Compressing the final blend (from Step (vii)) into tablets by using a tablet press; and
(ix) Preparing a film-coating suspension using the colored film-coating and purified water; and
(x) Spraying the coating suspension onto the tablet cores using a pan coater.

E34. The pharmaceutical formulation according to embodiment E33, wherein the process step (i) are performed as a sandwiching step with multiple alternating layers of microcrystalline cellulose and GDC-0077 (INAVOLISIB) being added to the bin blender.

E35. The pharmaceutical formulation according to embodiment E33 or E34, wherein the process step (i) is performed as a sandwiching step with multiple alternating layers of microcrystalline cellulose and GDC-0077 (INAVOLISIB) being added to the blender and wherein there is a first layer of microcrystalline cellulose (part A), a layer of GDC-0077 (INAVOLISIB), and a second layer of microcrystalline cellulose (part B).

E36. A pharmaceutical formulation according to embodiment E35, wherein step (ii) further comprises:
adding additional microcrystalline cellulose (part C) to the blend from Step (i), lactose, and the intragranular portion of sodium starch glycolate, before passing through the screening mill into the second bin blender.

E37. A pharmaceutical formulation according to any one of embodiments E1 to E36 wherein the GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) is free base GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide).

### EXAMPLES

The following examples are provided for illustration of the invention. They should not be considered as limiting the scope of the invention, but merely as being representative thereof.

### Example 1. Manufacturing of the Tablets

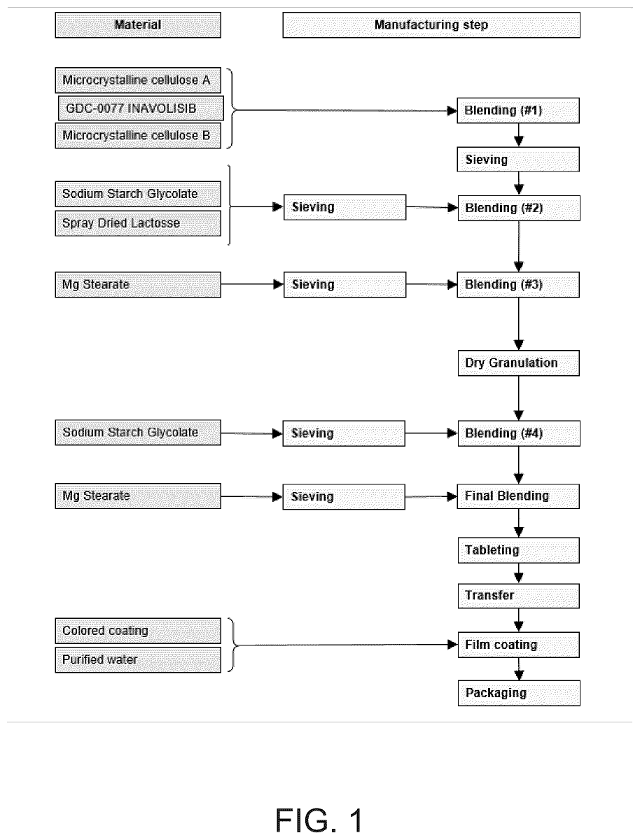

With reference to FIG. 1, a specific embodiment of present invention relates to a pharmaceutical formulation as described herein, obtainable by the process comprising the steps:
(i) Adding the microcrystalline cellulose (part) A, GDC-0077 (INAVOLISIB) and microcrystalline cellulose as a sandwiching step to a blender and blending;
(ii) Passing the blend from Step (i), lactose, and the intragranular portion of sodium starch glycolate, through a screening mill into a second blender;
(iii) Blending the powder blend from Step (ii);
(iv) Adding the intragranular portion of magnesium stearate and blending;
(v) Dry granulating the blend (from Step (iv)) using a roller compactor and passing through a screening mill;
(vi) Transferring the granules (from Step (v)) to a blender, adding the extra granular portion of sodium starch glycolate, and blending;
(vii) Adding the extragranular portion of magnesium stearate and blending;
(viii) Compressing the final blend (from Step (vii)) into tablets by using a tablet press; and
(ix) Preparing a film-coating suspension using the colored film-coating and purified water; and
(x) Spraying the coating suspension onto the tablet cores using a pan coater.

In this application blender is in particular a bin blender.

In one embodiment, step (i) is performed as a sandwiching step wherein multiple alternating layers of microcrystalline cellulose (in particular two layers part A and B)) and GDC-0077 (INAVOLISIB) are added to a blender. The sandwiching method is efficiently used to improve the blending efficiency and avoid that GDC-0077 (INAVOLISIB) stick to the equipment surface.

In one embodiment, the sandwiching step comprises adding a first layer of microcrystalline cellulose (part A), a layer of GDC-0077 (INAVOLISIB), and a second layer of microcrystalline cellulose (part B) are added to a blender.

In one embodiment, the sandwiching step comprises one layer of GDC-0077 (INAVOLISIB).

The sandwiching step has the advantage of improving processability of the formulation mixture and ensures a homogeneous mixture (blend uniformity).

### Example 2. Manufacturing of the Tablets

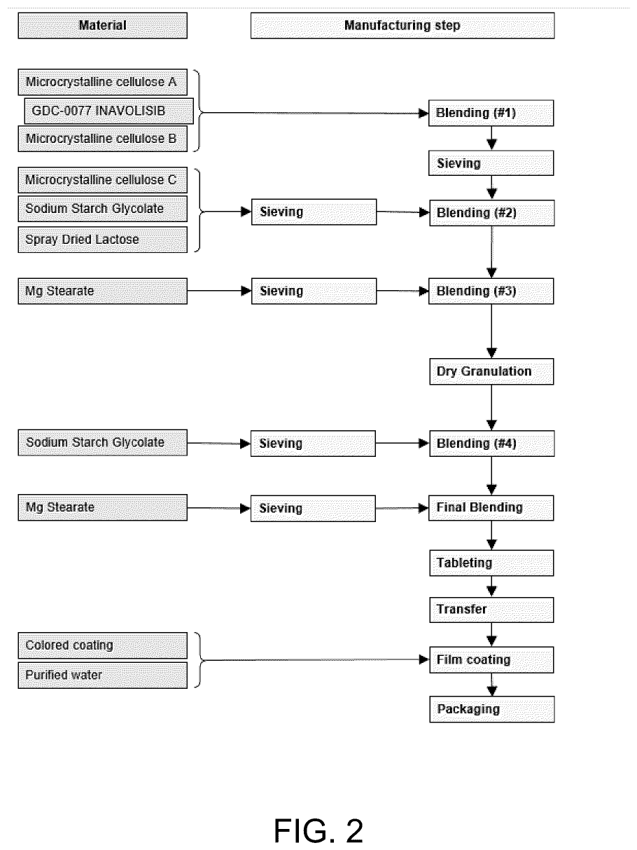

With reference to FIG. 2, a specific embodiment of present invention relates to a pharmaceutical formulation as described herein, obtainable by the process comprising the steps:
(i) Adding the microcrystalline cellulose (part) A, GDC-0077 (INAVOLISIB) and microcrystalline cellulose as a sandwiching step to a blender and blending;
(ii) Passing the blend from Step (i), microcrystalline cellulose (part C), lactose, and the intragranular portion of sodium starch glycolate, through a screening mill into a second blender;
(iii) Blending the powder blend from Step (ii);
(iv) Adding the intragranular portion of magnesium stearate and blending;
(v) Dry granulating the blend (from Step (iv)) using a roller compactor and passing through a screening mill;
(vi) Transferring the granules (from Step (v)) to a blender, adding the extra granular portion of sodium starch glycolate, and blending;
(vii) Adding the extragranular portion of magnesium stearate and blending;
(viii) Compressing the final blend (from Step (vii)) into tablets by using a tablet press; and
(ix) Preparing a film-coating suspension using the colored film-coating and purified water; and
(x) Spraying the coating suspension onto the tablet cores using a pan coater.

In this application blender in particular means a bin blender.

In some embodiments the process further comprises handsieving of intra- and extragranular magnesium stearate and of extragranular sodium starch glycolate prior addition.

### Example 3. Pharmaceutical Formulation

A pharmaceutical formulation that consists of:

| Components | Quantity per Unit Dose (% w/w) |
|---|---|
| Intra-Granular | |
| GDC-0077 (INAVOLISIB) | 3.0 |
| Microcrystalline Cellulose | 69.0 |
| Spray Dried Lactose | 23.0 |
| Sodium Starch Glycolate | 2.0 |
| Magnesium Stearate | 0.5 |
| Extra-Granular | |
| Sodium Starch Glycolate | 2.0 |
| Magnesium Stearate | 0.5 |
| Sub-Total (Core Tablet) | 100 |
| Colored film coating | 4.0 |

In particular the colored film coating is pink (3 mg GDC-0077 INAVOLISIB) or red (9 mg GDC-0077 INAVOLISIB).

### Example 4. Pharmaceutical Formulation

A pharmaceutical formulation composition of Inavolisib 3 mg and 9 mg film-coated tablets that consists of:

| | | | | |
|---|---|---|---|---|
| | | | 3 mg | 9 mg |
| | | | Amount per | Amount per |
| Component | Function | % | unit (mg) | unit (mg) |

Intragranular

| | | | | |
|---|---|---|---|---|
| INAVOLISIB (GDC-0077) | Active ingredient | 3.00 | 3.00 | 9.00 |
| Microcrystalline Cellulose | Filler | 69.00 | 69.00 | 207.00 |
| Spray Dried Lactose | Filler | 23.00 | 23.00 | 69.00 |
| Sodium Starch Glycolate | Disintegrant | 2.00 | 2.00 | 6.00 |
| Magnesium Stearate | Lubricant | 0.50 | 0.50 | 1.50 |

Extragranular

| | | | | |
|---|---|---|---|---|
| Sodium Starch Glycolate | Disintegrant | 2.00 | 2.00 | 6.00 |
| Magnesium Stearate | Lubricant | 0.50 | 0.50 | 1.50 |
| Weight core tablets | | 100.00 | 100.00 | 300.00 |
| Coating agent | Coating agent | +4% | 4.00 | 12.00 |
| | | weight | | |
| | | gain | | |
| Purified Water | Vehicle | q.s | 22.67 | 68.00 |
| Weight film-coated tablets | | | 104.00 | 312.00 |

In particular the colored film coating is pink (3 mg GDC-0077 INAVOLISIB) or red (9 mg GDC-0077 INAVOLISIB).

### Example 5. Pharmaceutical Formulation

A pharmaceutical formulation composition of Inavolisib 3 mg and 9 mg film-coated tablets that consists of:

### Formulation Composition of Inavolisib 3 mg and 9 mg Film-Coated Tablets

| | 3 mg | | 9 mg | |
|---|---|---|---|---|
| | Amount per | Amount per | Amount per | Amount per |
| Function | unit (mg) | unit (%) | unit (mg) | unit (%) |

Intragranular

| | | | | | |
|---|---|---|---|---|---|
| GDC-0077 | Active | 3.00 | 3.00 | 9.00 | 3.00 |
| INAVOLISIB | Ingredient | | | | |
| Microcrystalline | Filler | 20.00 | 20.00 | 50.00 | 16.67 |
| Cellulose, | | | | | |
| Part A^{a, b} | | | | | |
| Microcrystalline | Filler | 19.00 | 19.00 | 57.00 | 19.00 |
| Cellulose, Part B ^{b} | | | | | |
| Microcrystalline | Filler | 30.00 | 30.00 | 100.00 | 33.33 |
| Cellulose, Part C ^{b} | | | | | |
| Spray Dried | Filler | 23.00 | 23.00 | 69.00 | 23.00 |
| Lactose | | | | | |
| Sodium Starch | Disintegrant | 2.00 | 2.00 | 6.00 | 2.00 |
| Glycolate | | | | | |
| Magnesium | Lubricant | 0.50 | 0.50 | 1.50 | 0.50 |
| Stearate | | | | | |

Extragranular

| | | | | | |
|---|---|---|---|---|---|
| Sodium Starch | Disintegrant | 2.00 | 2.00 | 6.00 | 2.00 |
| Glycolate | | | | | |
| Magnesium | Lubricant | 0.50 | 0.50 | 1.50 | 0.50 |
| Stearate | | | | | |
| Weight core | | 100.00 | 100.00 | 300.00 | 100.00 |
| tablets | | | | | |
| Coating agent | Coating agent | 4.00 | +4% weight | 12.00 | +4% weight |
| | | | gain | | gain |
| Purified Water ^{c} | Vehicle | 22.67 | q.s. | 68.00 | q.s |

| | | | | |
|---|---|---|---|---|
| Weight film coated tablets | 104.00 | — | 312.00 | — |

^{a}The amount of API and MCC will be defined in accordance to the content of the API based on the CoA
^{b} The total amount of intragranular MCC in % is identical for 3 mg and 9 mg (i.e. 69.00%)
^{c} Removed during coating. The coating suspension is prepared at 15% w/w solids content. To be prepared in excess to allow proper functionality of the coating suspension system.

### Example 6. Pharmaceutical Formulation

A pharmaceutical formulation composition of Inavolisib 3 mg and 9 mg film-coated tablets that consists of:

| | | Quantity | Quantity |
|---|---|---|---|
| | | per Unit | per Unit |
| | | Dose (3 mg) | Dose (9 mg) |
| Components | Function | (mg/tablet) | (mg/tablet) |
| Tablet Core | | | |
| GDC-0077 INAVOLISIB | Active (API) | 3.0 | 9.0 |
| Microcrystalline Cellulose | Filler | 69.0 | 207.0 |
| Spray dried Lactose | Filler | 23.0 | 69.0 |
| Sodium Starch Glycolate | Disintegrant | 4.0 | 12.0 |
| Magnesium Stearate | Lubricant | 1.0 | 3.0 |
| Weight of Tablet Core | | 100.0 | 300.0 |
| Tablet core dimensions | | 6 mm round | 13 × 6.02 mm |
| | | bi-convex | oval, |
| | | 3.4 mm thick | 4.4 mm thick |
| Weight of Film-Coating | | 4.0 | 12.0 |
| Mixture | | | |
| Total Weight of | | 104.0 | 312.0 |
| Film-Coated Tablets | | | |

In particular the colored film coating is pink (3 mg GDC-0077 INAVOLISIB) or red (9 mg GDC-0077 INAVOLISIB).

### Example 7. Stability Data

GDC-0077 (INAVOLISIB) is sensitive to light, and a solid dosage pharmaceutical formulation comprising GDC-0077 (INAVOLISIB) is also sensitive to light.

In this study, a solid dosage pharmaceutical formulation comprising GDC-0077 (INAVOLISIB) with different colored film coatings were evaluated to compare the effects of film coating colors on light protection and guide the selection the appropriate film coating for formulation. The formulation used is shown in Table 3. The experimental conditions are as follows.

### A. ICH Light Exposure

A Suntest CPS+ light chamber was used with the following settings: Power: 765 W/m²; Temperature: 35 °C; Time: 7 hours (1×ICH, International Conference for Harmonization standard), 14 hours (2×ICH), 21 hours (3×ICH). Per ICH Q1B, 1×ICH light exposure is an overall illumination of not less than 1.2 million lux hours and an integrated near ultraviolet energy of not less than 200 watt hours/square meter.

1×ICH (8 Hours of Exposure was Actually Used in this Experiment)

For each of the three colored film coatings, 10 tablets were placed in separate glass scintillation vials respectively. The film-coated tablets were arranged in a single layer at the bottom of each scintillation vial. 10 uncoated tablets were also placed in a separate scintillation vial as a positive control. All scintillation vials were placed upright inside the light chamber.

In addition, 10 tablets for each of the three colored film coatings were placed in separate white HDPE containers with the lids on and containers upright.

### 2×ICH and 3×ICH

16 brown film-coated tablets were placed in two scintillation vials and exposed up to 3×ICH light conditions. The tablets were arranged in a single layer at the bottom of the scintillation vials. 16 uncoated tablets were also placed in separate scintillation vials as a positive control. All four scintillation vials were placed upright inside the light chamber.

### B. Direct Sunlight Exposure (During Daytime)

For each of the three colored film coatings, 30 coated tablets were placed in two glass scintillation vials laid on their side to create a single layer of tablets for 24 hours. 10 uncoated tablets were also placed in a separate scintillation vial to be used as a positive control. All scintillation vials were placed on their side on a windowsill where they were directly exposed to sunlight during the day, with the caps pointed away from the window. The windows of the labs have VEP 35 EnerLogic film on them.

In addition, 24 brown film-coated tablets were placed in three glass scintillation vials laid on their side to create a single layer of tablets up to 7 days. 16 uncoated tablets were also placed in two separate scintillation vials to be used as a positive control. All scintillation vials were placed on their side on a windowsill where they were directly exposed to sunlight during the day, with the caps pointed away from the window.

### C. Indoor Light

For each of the three colored film coatings, 30 coated tablets were placed in two glass scintillation vials laid on their side to create a single layer of tablets for 48 hours. 10 uncoated tablets were also placed in a separate scintillation vial to be used as a positive control. All scintillation vials were placed on their side on a bin in a fume hood, with the light turned on.

### D. Negative Control

A negative control was used for each condition by duplicate preps of unexposed coated tablets for each color of coating. No negative control was used for the uncoated tablets.

### Analytical Method

A validated HPLC method was used for the photostability study. The method conditions are described in Table 5 below.

TABLE 5
HPLC method

| Parameter | Condition | | |
|---|---|---|---|
| Column | Waters AcquityBEH Shield RP18, | | |
| | 2.1 × 150 mm, 1.7 um (P/N 186003376) | | |
| Column Temperature | 20 °C | | |
| Auto sampler Temperature | 5 °C | | |
| Flow Rate | 0.2 mL/min | | |
| Injection Volume | 2.5 μL | | |
| Detection Wavelength | UV at 215 nm, DAD: Bandwidth: 4 nm, | | |
| | Reference Wavelength: Off | | |
| | A = 5 mM Ammonium Acetate in Water with 0.020% (v/v) | | |
| | Glacial Acetic Acid, pH 4.8 | | |
| | B = 5 mM Ammonium Acetate in Methanol with 0.018% (v/v) | | |
| | Glacial Acetic Acid | | |
| Needle Wash | 50% Water/40% MeOH/10% Acetonitrile (v/v/v) | | |
| | Time (min) | % MPA | % MPB |
| Gradient Program | 0 | 97 | 3 |
| | 8 | 72 | 28 |
| | 30 | 59 | 41 |
| | 44 | 0 | 100 |
| | 46 | 0 | 100 |
| | 46.1 | 97 | 3 |
| | 61 | 97 | 3 |

The degradation products data by HPLC analysis for the tablets with three different coatings and uncoated tablets are summarized in Table 6.

TABLE 6
Degradation products in GDC-0077 (INAVOLISIB) coated
and uncoated tablets under various light conditions.

| Tablet | | | RRT 0.23 | RRT 0.65 | RRT 0.74 |
|---|---|---|---|---|---|
| Coating | Conditions | Timepoint | (area %) | (area %) | (area %) |
| Pink | Negative Control (not | T0 | 0.02 | ND | ND |
| | exposed) | | | | |
| | Light Chamber, in | 1x ICH | 0.08 | 0.03 | ND |
| | HDPE bottles | | | | |
| | Light Chamber, | 1x ICH | 0.08 | 0.03 | 0.02 |
| | exposed | | | | |
| | Indoor Light | 48 hours | 0.02 | ND | ND |
| | Direct Sunlight on | 24 hours | 0.06 | 0.02 | ND |
| | windowsill | | | | |
| Brown | Negative Control (not | T0 | 0.01 | ND | ND |
| | exposed) | | | | |
| | Light Chamber, in | 1x ICH | 0.03 | 0.02 | ND |
| | HDPE bottles | | | | |
| | Light Chamber, | 1x ICH | 0.03 | ND | ND |
| | exposed | | | | |
| | Indoor Light | 48 hours | 0.02 | 0.03 | ND |
| | Direct Sunlight on | 24 hours | 0.03 | 0.03 | ND |
| | windowsill | | | | |
| Yellow | Negative Control (not | T0 | 0.01 | 0.03 | ND |
| | exposed) | | | | |
| | Light Chamber, in | 1x ICH | 0.06 | 0.05 | 0.02 |
| | HDPE bottles | | | | |
| | Light Chamber, | 1x ICH | 0.05 | 0.04 | 0.02 |
| | exposed | | | | |
| | Indoor Light | 48 hours | 0.03 | 0.03 | ND |
| | Direct Sunlight on | 24 hours | 0.10 | 0.07 | ND |
| | windowsill | | | | |
| Uncoated | Light Chamber, | 1x ICH | 0.11 | 0.04 | 0.09 |
| | exposed | | | | |
| | Indoor Light | 48 hours | 0.04 | 0.03 | 0.03 |
| | Direct Sunlight on | 24 hours | 0.10 | 0.04 | 0.09 |
| | windowsill | | | | |

ND: none detected. The LOQ of the method is 0.05%. Values lower than 0.05% are reported here for comparative purposes.

The uncoated tablets showed the largest increase in degradation products under all light conditions. The pink and yellow film-coated tablets showed moderate increase in degradation products after 1×ICH light exposure and direct sunlight exposure. The brown film-coated tablets showed the least increase in degradation under all light conditions.

The white HDPE bottles did not appear to provide light protection, as the levels of degradation were the same whether the tablets were exposed to light directly or they were inside the HDPE bottles.

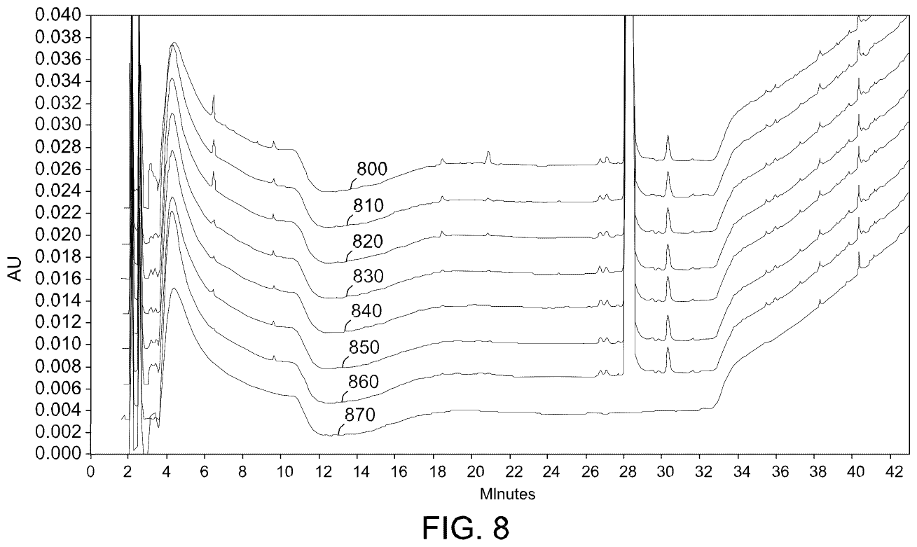

The chromatograms overlay for the tablets before and after 1×ICH light exposure is shown in FIG. 8. The key for each chromatogram depicted in FIG. 8 is as follows:
Uncoated tablets (light exposed): 800
Yellow Tablets (light exposed): 810
Pink Tablets (light exposed): 820
Brown Tablets (light exposed): 830
Yellow Tablets (not exposed): 840
Pink Tablets (not exposed): 850
Brown Tablets (not exposed): 860
Blank: 870

The degradation products from HPLC analysis of the brown film-coated tablets and uncoated tablets subject to additional light exposure are summarized in Table 7. The brown film coating continued to provide light protection that resulted in much lower degradation even at 2× and 3×ICH light exposure and up to 7 days of direct sunlight exposure, compared with uncoated tablets.

TABLE 7
Degradation products in GDC-0077 (INAVOLISIB) brown film-coated
tablets and uncoated tablets under extended light exposure conditions
color coated tablets were unchanged after exposure to all the
light conditions tested (ICH, sunlight, indoor light).

| Tablet | | | RRT 0.23 | RRT 0.34 | RRT 0.65 | RRT 0.74 |
|---|---|---|---|---|---|---|
| Coating | Conditions | Timepoint | (area %) | (area %) | (area %) | (area %) |
| Brown | Negative Control | T0 | 0.01 | 0.02 | ND | ND |
| | Light Chamber, | 2x ICH | 0.04 | 0.02 | 0.02 | ND |
| | exposed | 3x ICH | 0.04 | 0.02 | 0.02 | ND |
| | Direct Sunlight | 3 days | 0.04 | 0.02 | 0.02 | 0.01 |
| | on windowsill | 7 days | 0.04 | 0.02 | ND | ND |
| Uncoated | Light Chamber, | 2x ICH | 0.13 | 0.04 | 0.07 | 0.05 |
| | exposed | 3x ICH | 0.17 | 0.04 | 0.09 | 0.06 |
| | Direct Sunlight | 3 days | 0.14 | 0.04 | 0.05 | 0.07 |
| | on windowsill | 7 days | 0.19 | 0.05 | 0.08 | 0.07 |

### Visual Observations

### Exterior Color

The color of the exterior of the three color coated tablets were unchanged after exposure to all the light conditions tested (ICH, sunlight, indoor light).

### Interior Color

On examination of the interior colour, the presence of yellow color in the white tablet core is indicative of decomposition or change on exposure to light of the active GDC-0077 (INAVOLISIB) and correlates with lower assay of the active. A white tablet core is indicative of low or no decomposition or change on exposure to light of the active GDC-0077 (INAVOLISIB) and correlates with a relatively higher assay of the active.

### 1×ICH Light Exposure

After 1×ICH light exposure, the tablets with three different color coatings were cut open to inspect any color change inside the tablets. The core of the yellow and pink tablets under the coating turned yellow, whether the tablets were directly exposed or inside white HDPE bottles. Under the same conditions, the core of the brown tablets under the coating remained white after ICH exposure.

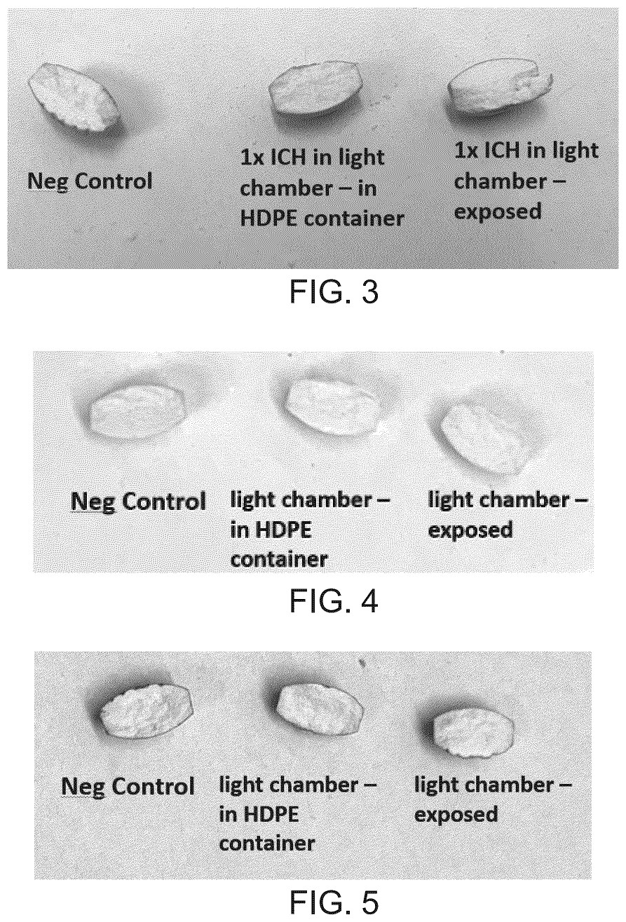

The pictures of tablets with different color coatings before and after 1×ICH light exposure are shown in FIGS. 3-5.

### 2× and 3×ICH Light Exposure

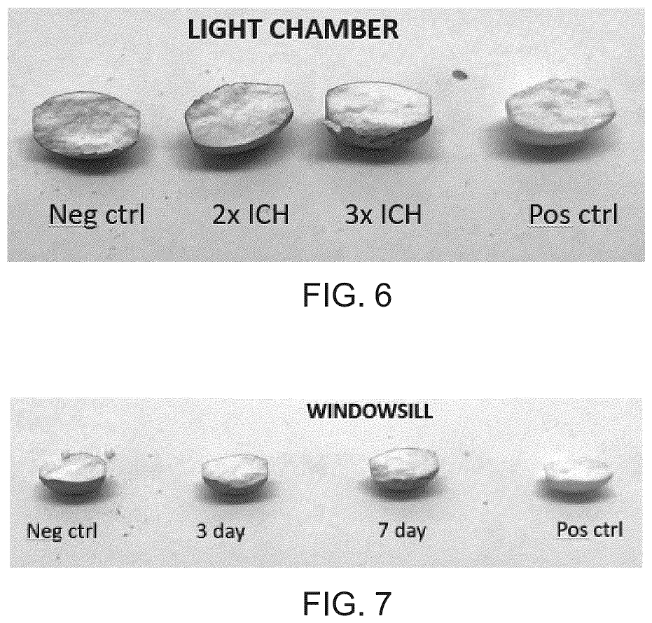

After 2× and 3×ICH light exposure, the brown film-coated tablets were cut open to inspect any color change inside the tablets. Slight yellow hues were observed under the film-coating, as shown in FIG. 6.

### Direct Sunlight Exposure

After 3 and 7 days of direct sunlight exposure on the windowsill, the brown film-coated tablets were cut open to inspect any color change inside the tablets. Slight yellow hues were observed under the film-coating, as shown in FIG. 7.

ICH and other light conditions

Table 7 from Anderson, N. H., and Stephen J. Byard. “Photostability testing: Design and interpretation of tests on new drug substances and dosage forms.” Photostability of drugs and drug formulations. CRC Press, Boca Raton, Fl, 1996, 137-172 shows the light levels under various light conditions. The ICH light exposure of not less than 1.2 million lux hours and an integrated near ultraviolet energy of not less than 200 watt hours/square meter is similar to two days of light exposure on a sunny windowsill.

The data in Table 6 and Table 7 show that degradation levels after 1×ICH light exposure are similar to those after 1-2 days on a sunny windowsill. The photostability data for GDC-0077 (INAVOLISIB) tablets on sunny windowsill are consistent with and supplemental to the data under ICH light exposure.

TABLE 8
Light exposure under various condition

| | | | Days Under |
|---|---|---|---|
| | | | Artificial Light at |
| | Days on Sunny | Days 1 m from | 500 klx (24 hour |
| | Window Sill | Window | day) |
| Exposure (klx h) | | | |
| 1200 | 2-3 | ca. 12 | 100 |
| 2400 | 4-6 | ca. 24 | 200 |
| 3600 | 8-12 | ca. 36 | 300 |
| UV Exposure | | | |
| Wh m⁻² | | | |
| 200 | 1-2 | ca. 50 | |
| 500 | 3-5 | ca. 125 | |

Among the three film coatings tested, brown coating provided the best light protection for GDC-0077 (INAVOLISIB) tablets. There is no color change on the exterior or in the tablet core under the film coatings after 1×ICH light exposure. The brown tablets also showed the least increase in degradation products among the tablets coated with three different colors. No degradation product >0.05% was observed in the brown tablets after various light exposure conditions. Together with the intragranular and extragranular excipients provides the desired stability and processability.

The yellow and pink film coated tablets maintained their exterior color, but the tablet core under the coating turned yellow after light exposure at 1×ICH conditions. There were also higher levels of degradation products after 1×ICH light exposure, compared with brown film-coated tablets.

### Hunterlab Measurements

The tablets and API were evaluated for appearance at each time point.

This was performed visually with the Munsell Book of color and by measuring reflected color with the help of a spectrophotometer (UltraScan PRO-Hunterlab)

The tablets were evaluated from the exterior but also in the interior (only with the Munsell Book for the interior of the tablets). This allowed tracking of any slight color change that starts to happen but that was still masked by the coating.

### Principle of Hunterlab System:

The UltraScan Pro is a spectrophotometer with mathematical modeling of all the colors perceived by the human being (3-dimensional color space L*a*b*).

The evaluation of color change is performed based on the ΔE₂₀₀₀ value.

ΔE₂₀₀₀ is the distance between points within the 3-dimensional L*a*b* color space where:
L*=lightness/darkness value
+=lighter −=darker
a*=red/greenaxis
+=redder −=greener
b*=yellow/blueaxis
+=yellower −=bluer

This distance correlates to human perception.

As used herein and based on normal use of this system, no color change can be concluded if the ΔE2000 value≤2.

The tablets were also analyzed regarding the content and degradation products. The specification criteria was used to evaluate the samples. Regarding the appearance due to the broad range of colors included in the specifications, the actual appearance of the tablet was required to be properly described and recorded. This allows a better understanding of how the changes are happening.

There is a high chance that the formed degradation products associated with the color change do affect the quality of the product. Therefore, it is important to record accurately the difference between internal and external of appearance. In this view, an exterior unchanged appearance might be acceptable even if the interior shows evident color change.

The pink and red colored tablets formed in Example 5 were tested and showed a Hunterlab measurement ΔE₂₀₀₀≤1, meaning that no color change was observed. This is indicative of an acceptably low degradation of the exterior appearance.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

The features of any specific embodiment described herein may be combined with features from any other specific embodiment(s) insofar as they are compatible and combination is appropriate. It is particularly envisaged that features that are described individually in relation to a “specific embodiment” may be combined with one or more other features from specific embodiments set out elsewhere herein to provide a new embodiment of the invention.

All documents referred to herein are hereby incorporated by reference in their entirety.

1-34. (canceled)

35. A pharmaceutical formulation in the form of a solid dosage form comprising GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) having the formula: 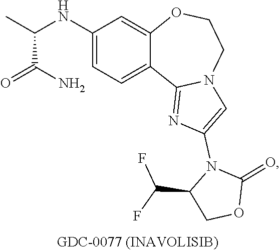 [SMILES: C[C@H](Nc1ccc2c(c1)OCCn1cc(N3C(=O)OC[C@H]3C(F)F)nc1-2)C(N)=O]
or a pharmaceutically acceptable salt thereof, wherein the pharmaceutical formulation comprises (i) 3 mg or 9 mg of GDC-0077 (INAVOLISIB), or a pharmaceutically acceptable salt thereof, and (ii) a colored coating that is a water-soluble film coating that comprises a colored pigment, wherein the colored pigment comprises one or more iron oxide(s), and wherein the one or more iron oxide(s) is iron oxide (red), or a combination of iron oxide (red) and iron oxide (yellow).

36. The pharmaceutical formulation according to claim 35, wherein the solid dosage form is an immediate release tablet for oral administration.

37. The pharmaceutical formulation according to claim 35, wherein the pharmaceutical formulation comprises (iii) one or more intragranular excipients, and (iv) one or more extragranular excipients.

38. The pharmaceutical formulation according to claim 37, wherein the intragranular excipients are selected from the group consisting of binders, disintegrants, glidants, fillers, and lubricants.

39. The pharmaceutical formulation according to claim 38, wherein the intragranular excipients are selected from the group consisting of fillers, disintegrants, and lubricants.

40. The pharmaceutical formulation according to claim 37, wherein the extragranular excipients are selected from the group consisting of binders, disintegrants, glidants, fillers, and lubricants.

41. The pharmaceutical formulation according to claim 40, where the extragranular excipients are selected from the group consisting of disintegrants and lubricants.

42. The pharmaceutical formulation according to claim 37, wherein there are two intragranular fillers which are microcrystalline cellulose and spray dried lactose.

43. The pharmaceutical formulation according to claim 37, wherein there is one intragranular disintegrant which is sodium starch glycolate.

44. The pharmaceutical formulation according to claim 37, wherein there is one intragranular lubricant which is magnesium stearate.

45. The pharmaceutical formulation according to claim 37, wherein there is one extragranular disintegrant which is sodium starch glycolate.

46. The pharmaceutical formulation according to claim 37, wherein there is one extragranular lubricant which is magnesium stearate.

47. The pharmaceutical formulation according to claim 35, wherein the colored coating consists of:

| | |
|---|---|
| Polyvinylalcohol (partially hydrolyzed) | about 40.0% |
| Titanium dioxide | about 10.0% |
| Macrogol/Polyethylene glycol | about 20.2% |
| Talc | about 14.8% |
| Iron oxide (red) | about 15.0% |

or

| | |
|---|---|
| Polyvinylalcohol (partially hydrolyzed) | about 40.0% |
| Titanium dioxide | about 22.1% |
| Macrogol/Polyethylene glycol | about 20.2% |
| Talc | about 14.8% |
| Iron oxide (red) | about 1.5% |
| Iron oxide (yellow) | about 1.4% |

48. The pharmaceutical formulation according to claim 47, wherein the colored coating consists of:

| | |
|---|---|
| Polyvinylalcohol (partially hydrolyzed) | about 40.0% |
| Titanium dioxide | about 10.0% |
| Macrogol/Polyethylene glycol | about 20.2% |
| Talc | about 14.8% |
| Iron oxide (red) | about 15.0% |

49. The pharmaceutical formulation according to claim 47, wherein the colored coating consists of:

| | |
|---|---|
| Polyvinylalcohol (partially hydrolyzed) | about 40.0% |
| Titanium dioxide | about 22.1% |
| Macrogol/Polyethylene glycol | about 20.2% |
| Talc | about 14.8% |
| Iron oxide (red) | about 1.5% |
| Iron oxide (yellow) | about 1.4% |

50. The pharmaceutical formulation according to claim 35, wherein the colored coating is Opadry II™.

51. The pharmaceutical formulation according to claim 35, wherein the weight of the film-coating mixture per tablet is in the range of about 3 mg to about 15 mg.

52. The pharmaceutical formulation according to claim 51, wherein the weight of the film-coating mixture per tablet is about 4 mg or about 12 mg.

53. The pharmaceutical formulation according to claim 35, that consists of:

| | Quantity per Unit Dose |
|---|---|
| Components | % w/w |
| Intra-Granular | |
| GDC-0077 (INAVOLISIB) | 3.0 |
| Microcrystalline Cellulose | 69.0 |
| Spray Dried Lactose | 23.0 |
| Sodium Starch Glycolate | 2.0 |
| Magnesium Stearate | 0.5 |
| Extra-Granular | |
| Sodium Starch Glycolate | 2.0 |
| Magnesium Stearate | 0.5 |
| Sub-Total (Core Tablet) | 100 |
| Colored film coating | 4.0 |

54. The pharmaceutical formulation according to claim 35, obtainable by the process comprising the steps of:
(i) blending GDC-0077 (INAVOLISIB) with one or more intragranular excipients;
(ii) dry granulating the blend resulting from step (i);
(iii) blending the granulated product of step (ii) with one or more extragranular excipients;
(iv) tableting the blend resulting from step (iii) into tablets; and
(v) applying the coloured coating formulation to the exterior of the tablets.

55. The pharmaceutical formulation according to claim 54, wherein the particles of the GDC-0077 (INAVOLISIB) in step (i) have a D50 value between 10 and 132 μm and D90 value below 264 μm.

56. The pharmaceutical formulation according to claim 35, obtainable by the process comprising the steps of:
(i) Adding microcrystalline cellulose (part A), GDC-0077 (INAVOLISIB) and microcrystalline cellulose (part B) as a sandwiching step to a blender and blending;
(ii) Passing the blend from Step (i), lactose, and an intragranular portion of sodium starch glycolate, through a screening mill into a second blender;
(iii) Blending the powder blend from Step (ii);
(iv) Adding an intragranular portion of magnesium stearate and blending;
(v) Dry granulating the blend (from Step (iv)) using a roller compactor and passing through a screening mill;
(vi) Transferring the granules (from Step (v)) to a blender, adding an extra granular portion of sodium starch glycolate, and blending;
(vii) Adding an extragranular portion of magnesium stearate and blending;
(viii) Compressing the final blend (from Step (vii)) into tablets by using a tablet press; and
(ix) Preparing a film-coating suspension using the colored film-coating and purified water; and
(x) Spraying the coating suspension onto the tablet cores using a pan coater.

57. A pharmaceutical formulation according to claim 35 wherein the GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide) is free base GDC-0077 (INAVOLISIB) ((S)-2-((2-((S)-4-(difluoromethyl)-2-oxooxazolidin-3-yl)-5,6-dihydrobenzo[f]imidazo[1,2-d][1,4]oxazepin-9-yl)amino)propanamide).

58. A method of treating cancer comprising administering to a patient in need thereof a pharmaceutical formulation according claim 35.

59. The method according to claim 58 wherein the cancer is a cancer associated with PIK3CA mutations.

60. The method according to claim 58 wherein the cancer is breast cancer.

61. The method according to claim 60, wherein the breast cancer is ½L PIK3CAmt HR+ mBC.
